(19) United States
(12) Patent Application Publication
(10) Pub. No.: US 2016/0375101 A1
(43) Pub. Date: Dec. 29, 2016

(54) Methods of Using Interleukin-10 for Treating Diseases and Disorders

(71) Applicant: ARMO BIOSCIENCES, INC., Redwwod City, CA (US)

(72) Inventor: Martin Oft, Palo Alto, CA (US)

(21) Appl. No.: 15/110,925

(22) PCT Filed: Jan. 9, 2015
(86) PCT No.: PCT/US15/10856

§ 371 Date: Jul. 11, 2016

Related U.S. Application Data
(60) Provisional application No. 61/927,893, filed on Jan. 15, 2014.

(51) Int. Cl. A61K 38/20; G01N 33/50; A61K 47/48; G01N 33/92; A61K 9/00; A61K 45/06

(52) U.S. Cl. CPC A61K 38/2066; A61K 9/0019; A61K 45/06; A61K 47/48215; G01N 33/92; G01N 33/5088; G01N 2333/5428; G01N 2800/52; G01N 2800/323; G01N 2800/325; G01N 2800/32; G01N 2800/328

(57) ABSTRACT

Methods for determining dosing parameters associated with subcutaneous administration of IL-10 useful for the treatment and/or prevention of a cholesterol-related disease, disorder or condition, and pharmaceutical compositions associated therewith, are provided herein. Certain embodiments are directed to means for establishing a therapeutic range of an IL-10 agent in a subject. In some embodiments, particular parameters (e.g., hematological parameters) are monitored to provide an indication of the upper limit of the therapeutic range such that any untoward adverse effects are avoided.

### CROSS-REFERENCE TO RELATED APPLICATION

This application claims priority benefit of U.S. provisional application Ser. No. 61/927,893, filed Jan. 15, 2014, which application is incorporated herein by reference in its entirety.

### FIELD OF THE INVENTION

This invention relates to methods of using IL-10 and related agents in the treatment or prevention of hypercholesterolemia and a diverse array of related diseases and disorders.

### INTRODUCTION

The cytokine interleukin-10 (IL-10) is a pleiotropic cytokine that regulates multiple immune responses through actions on T cells, B cells, macrophages, and antigen presenting cells (APC). IL-10 may suppress immune responses by inhibiting expression of IL-1α, IL-1β, IL-6, IL-8, TNF-α, GM-CSF and G-CSF in activated monocytes and activated macrophages, and it also suppresses IFN-γ production by NK cells. Although IL-10 is predominantly expressed in macrophages, expression has also been detected in activated T cells, B cells, mast cells, and monocytes. In addition to suppressing immune responses, IL-10 exhibits immuno-stimulatory properties, including stimulating the proliferation of IL-2—and IL-4—treated thymocytes, enhancing the viability of B cells, and stimulating the expression of MHC class II.

Human IL-10 is a homodimer that becomes biologically inactive upon disruption of the non-covalent interactions between the two monomer subunits. Data obtained from the published crystal structure of IL-10 indicates that the functional dimer exhibits certain similarities to IFN-γ (Zdanov et al, (1995) Structure (Lond) 3:591-601).

As a result of its pleiotropic activity, IL-10 has been linked to a broad range of diseases, disorders and conditions, including inflammatory conditions, immune-related disorders, fibrotic disorders, metabolic disorders and cancer. Clinical and pre-clinical evaluations with IL-10 for a number of such diseases, disorders and conditions have solidified its therapeutic potential. Moreover, pegylated IL-10 has been shown to be more efficacious than non-pegylated IL-10 in certain therapeutic settings. In addition, efforts to target IL-10 agents to specific cell types have been explored (see, e.g., Rachmawati, H. (May 2007) Drug Met. Dist. 35(5):814-21).

In view of the prevalence of metabolic diseases, disorders and conditions, such as hypercholesterolemia, and their associated morbidity, alternative treatment regimens and dosing parameters that optimize efficacy, patient tolerance and the like would be of tremendous value.

### SUMMARY

The present disclosure pertains to methods and compositions comprising IL-10 agents (e.g., PEG-IL-10) and their association with the treatment and/or prevention of various diseases, disorders and conditions, and/or the symptoms thereof. More particularly, the present disclosure relates to, for example, methods for determining amounts of an IL-10 agent (e.g., PEG-IL-10) for subcutaneous administration that are therapeutically useful for the treatment and/or prevention of a cholesterol-related disease, disorder or condition in a subject (e.g., a human); dosing regimens comprising the administration of such amounts; and pharmaceutical compositions comprising the amounts of the IL-10 agents. Certain embodiments are directed to means for establishing a therapeutic range of an IL-10 agent in a subject. In some embodiments, particular parameters (e.g., hematological parameters) are monitored to provide an indication of the upper limit of the therapeutic range such that any untoward adverse effects are avoided.

Historically, subjects administered IL-10 agents for the treatment or prevention of various diseases, disorders, and conditions (e.g., cancer-related disorders, immune/inflammatory-related disorders, and metabolic-related disorders) have received the agents parenterally, including via intravenous and subcutaneous injection. As one might expect, compared to IV dosing, SC administration of IL-10 has been shown to result in extended exposure at the expense of peak exposure. In the context of cholesterol lowering, the inventors have made the surprising discovery that SC administration of a particular dose of IL-10 is pharmacodynamically more active than a much larger dose administered IV, even when similar serum trough concentrations are compared.

As discussed hereafter, optimization of dosing parameters and regimens involves, for example, the assessment of an IL-10 agent's pharmacokinetic and pharmacodynamic parameters associated with absorption, distribution, metabolism, and excretion (“ADME”), taking into consideration the route of administration and other factors. It is understood that, unless indicated otherwise herein, terms related to ADME and other parameters are intended to have their ordinary accepted meanings in the relevant scientific fields. By way of example, the terms “serum half-life” or “t_{1/2},” refer to elimination half-life (i.e., the time at which the serum concentration of an agent has reached one-half of its initial or maximum value). As used herein, reference to serum concentration is meant to include plasma concentration, and vice versa.

Hypercholesterolemia itself is generally asymptomatic. However, chronic elevation of serum cholesterol contributes to formation of atheromatous plaques in the arteries. Relatively small plaques may rupture and cause a clot to form and obstruct blood flow. By comparison, larger plaques can result in arterial stenosis or occlusion of the involved arteries. A sudden occlusion of a coronary artery results in a myocardial infarction, whereas an occlusion of an artery supplying the brain can result in a stroke.

Gradual development of the stenosis or occlusion that causes a progressive reduction in the blood supply to the tissues and organs frequently results in impairment of the activity thereof. Tissue ischemia may manifest as one or more symptoms. For example, temporary ischemia of the brain (a transient ischemic attack) may manifest as temporary loss of vision, dizziness, or impairment of balance, aphasia, paresis and paresthesia. Insufficient blood supply to the heart may manifest as chest pain; ischemia of the eye may manifest as transient visual loss in one eye; and insufficient blood supply to the legs may manifest as calf pain.

According to the lipid hypothesis, abnormal cholesterol levels (generally higher concentrations of LDL particles and lower concentrations of functional HDL particles) in the blood are strongly associated with cardiovascular disease due to promotion of atheroma development in arteries (atherosclerosis). As high circulating LDL concentrations have been linked to atheroma formation, LDL is often referred to as “bad cholesterol”; in contrast, high concentrations of HDL can remove cholesterol from cells, diminishing atheroma formation, and thus HDL is often referred to as “good cholesterol”. However, recent evidence suggests that total cholesterol is the most relevant indicator of cardiovascular abnormalities.

When dietary restrictions alone are insufficient in addressing hypercholesterolemia, one or more hypolipidemic agents (e.g., statins, fibrates, cholesterol absorption inhibitors, nicotinic acid derivatives and bile acid sequestrants) are often introduced. If pharmacological therapy is unsuccessful, several extreme procedures have been utilized (e.g., apheresis-based treatment). According to the teachings of the present disclosure, the IL-10—related agents described herein provide an alternative therapeutic modality that can be substituted for, or combined with, hypolipidemic agents such as those described herein.

There are a limited number of disclosures reporting the use of IL-10 in cholesterol lowering. U.S. Pat. No. 5,945,097 reported that normal, healthy human subjects administered 8 μg/kg rhIL-10 subcutaneously daily for seven days exhibited an approximately 43% reduction in total serum cholesterol compared to subjects receiving placebo; however, assessments were not made in subjects with hypercholesterolemia. In addition, previous reports did not describe the relative effect of IL-10 on different types of cholesterol (e.g., LDL and HDL). Moreover, the identification of optimized dosing parameters for the treatment of hypercholesterolemia (e.g., the therapeutic range of and IL-10 agent in a subject) and other lipid-related disorders have not been described.

As discussed further hereafter, human IL-10 is a homodimer and each monomer comprises 178 amino acids, the first 18 of which comprise a signal peptide. Particular embodiments of the present disclosure comprise mature human IL-10 polypeptides lacking the signal peptide (see, e.g., U.S. Pat. No. 6,217,857), or mature human PEG-IL-10. In further particular embodiments, the IL-10 agent is a variant of mature human IL-10. The variant may exhibit activity less than, comparable to, or greater than the activity of mature human IL-10; in certain embodiments, the activity is comparable to or greater than the activity of mature human IL-10.

The terms “IL-10”, “IL-10 polypeptide(s),” “IL-10 agent(s)” and the like are intended to be construed broadly and include, for example, human and non-human IL-10—related polypeptides, including homologs, variants (including muteins), and fragments thereof, as well as IL-10 polypeptides having, for example, a leader sequence (e.g., the signal peptide), and modified versions of the foregoing. In further particular embodiments, the terms “IL-10”, “IL-10 polypeptide(s), and “IL-10 agent(s)” are agonists. Particular embodiments relate to pegylated IL-1β, which is also referred to herein as “PEG-IL-10”. The present disclosure also contemplates nucleic acid molecules encoding the foregoing.

In particular embodiments of the present disclosure, a therapeutically effective amount of IL-10 is an amount that results in reduction of less than 5%, about 5%, about 6%, about 7%, about 8%, about 9%, about 10%, about 11%, about 12%, about 13%, about 14%, about 15%, or more than 15% of total serum cholesterol in a subject 48 hours after administration of a single subcutaneous dose. In some embodiments of the present disclosure, a therapeutically effective amount is a dose of IL-10 that results in reduction of about 8%, about 9%, about 10%, about 11% or about 12% of total serum cholesterol in a subject 48 hours after administration of a single subcutaneous dose. It is to be understood that the 48 hour post-dose time point is just one of many possible benchmarks for determining whether a dose is therapeutically effective. By way of example, but not limitation, time points of less than 24 hours, about 24 hours, about 26 hours, about 28 hours, about 30 hours, about 32 hours, about 34 hours, about 36 hours, about 38 hours, about 40 hours, about 42 hours, about 44 hours, about 45 hours, about 46 hours, about 47 hours, about 48 hours, about 49 hours, about 50 hours, about 51 hours, about 52 hours, about 53 hours, about 54 hours, about 55 hours, or more than 55 hours may be used in determining whether an amount (e.g., a dose) is therapeutically effective.

The present disclosure contemplates the subcutaneous administration of any amount to a subject (e.g., a human) that achieves a desired reduction in total serum cholesterol at a particular time point. By way of example, but not limitation, a reduction of, e.g., at least 10% of total serum cholesterol determined, e.g., 48 hours post-dose may be achieved by an amount of IL-10 of about 1.0 μg/kg, about 2.5 μg/kg, about 5.0 μg/kg, about 10 μg/kg, about 25 μg/kg about 50 μg/kg, or more than 50 μg/kg of the body weight of a subject (e.g., a human). In particular embodiments, a therapeutically effective amount is about 2.0 μg/kg, about 2.25 μg/kg about 2.5 μg/kg, about 2.75 μg/kg about 3.0 μg/kg, about 3.25 μg/kg about 3.5 μg/kg, about 3.75 μg/kg, about 4.0 μg/kg, about 4.25 μg/kg, about 4.5 μg/kg, about 4.75 μg/kg, or about 5.0 μg/kg. The aforementioned amounts may be administered, for example, every 12 hours, every 24 hours, every 36 hours, every 48 hours, every 60 hours or every 72 hours; in certain embodiments of the present disclosure, the amount is administered every 48 hours. In some embodiments, the therapeutically effective amount is an amount that achieves a desired reduction in total serum cholesterol at a particular time point in the absence of an unacceptable indicia of an IL-10 agent—associated toxicity in the subject. Examples of unacceptable indicia of an IL-10 agent—associated toxicity are described elsewhere herein.

The aforementioned dosing parameters can form the basis of other dosing regimens. For example, as indicated in Example 1, subcutaneous administration of >2.5 μg/kg every 48 hours to a human subject achieved a reduction in total serum cholesterol of about 10%. By way of example, these data can be extrapolated such that about 16 μg/kg of IL-10 is administered weekly to the subject.

In the examples in the Experimental section, reduction of total serum cholesterol by a defined amount at a defined time point is described in the context of non-pegylated IL-10. The present disclosure also contemplates the use of the teachings described herein with pegylated hIL-10. The skilled artisan is able to translate the dosing parameters used with non-pegylated IL-10 to those useful for the administration of pegylated IL-10. By way of illustration, but not limitation, when the subject is a human, non-pegylated hIL-10 may be administered at a dose greater than 0.5 μg/kg/day, greater than 1.0 μg/kg/day, greater than 2.5 μg/kg/day, greater than 5 μg/kg/day, greater than 7.5 μg/kg, greater than 10.0 μg/kg, greater than 12.5 μg/kg, greater than 15 μg/kg/day, greater than 17.5 μg/kg/day, greater than 20 μg/kg/day, greater than 22.5 μg/kg/day, greater than 25 μg/kg/day, greater than 30 μg/kg/day, or greater than 35 μg/kg/day. Such non-pegylated hIL-10 dosing parameters may translate into dosing parameters with pegylated hIL-10 comprising a relatively small PEG (e.g., 5 kDa mono-di-PEG-hIL-10) of a dose greater than 0.5 μg/kg/day, greater than 0.75 μg/kg/day, greater than 1.0 μg/kg/day, greater than 1.25 μg/kg/day, greater than 1.5 μg/kg/day, greater than 1.75 μg/kg/day, greater than 2.0 μg/kg/day, greater than 2.25 μg/kg/day, greater than 2.5 μg/kg/day, greater than 2.75 μg/kg/day, greater than 3.0 μg/kg/day, greater than 3.25 μg/kg/day, greater than 3.5 μg/kg/day, greater than 3.75 μg/kg/day, greater than 4.0 μg/kg/day, greater than 4.25 μg/kg/day, greater than 4.5 μg/kg/day, greater than 4.75 μg/kg/day, or greater than 5.0 μg/kg/day.

In certain aspects, the present disclosure contemplates a method for determining whether an amount of an IL-10 agent administered subcutaneously is therapeutically effective for treating a cholesterol-related disease, disorder or condition in a subject, comprising a) establishing a baseline concentration of total serum cholesterol in the subject, b) administering subcutaneously to the subject a test amount of an IL-10 agent of at least 1 μg/kg, and c) measuring the total serum cholesterol reduction in the subject at least 48 hours after step b), wherein a reduction in total serum cholesterol of at least 10% and the absence of an unacceptable indicia of an IL-10 agent—associated toxicity in the subject, indicates a therapeutically effective amount.

As used herein, the term “baseline” is meant to provide a reference point for comparing or calculating another parameter. In particular embodiments, reference to a baseline concentration indicates that the subject has no measurable IL-10 serum concentration. Examples of unacceptable indicia of an IL-10 agent—associated toxicity in the subject, which can be viewed as the upper limit of a subject's therapeutic range, are discussed elsewhere herein, and include various hematological parameters.

Other aspects of the present disclosure contemplate a method for achieving an amount of an IL-10 agent for subcutaneous administration sufficient for the treatment of a cholesterol-related disease, disorder or condition in a subject, comprising a) establishing a baseline concentration of total serum cholesterol in the subject, b) administering subcutaneously to the subject a first test amount of an IL-10 agent, wherein the first test amount is at least 1 μg/kg, c) measuring the total serum cholesterol reduction in the subject at least 48 hours after administration of the first test amount, and wherein a reduction of at least 10% in the subject indicates that the first test amount is sufficient for the treatment of a cholesterol-related disease, disorder or condition in a subject, wherein a reduction of less than 10% in the subject indicates that the first test amount is suboptimal, whereafter i) a second test amount greater than the first test amount is subcutaneously administered to the subject, wherein the subject has no measurable IL-10 serum concentration, and ii) the total serum concentration in the subject is measured at least 48 hours after the second test amount; wherein steps i) and ii) are repeated until a reduction of at least 10% is achieved indicating that the test amount is sufficient for the treatment of a cholesterol-related disease, disorder or condition.

In reference to the methods set forth above, in some embodiments the measuring of total serum cholesterol reduction can be conducted at any other time that is appropriate under the circumstances. For example, it can be conducted at least 12 hours after step b), at least 24 hours after step b), at least 72 hours after step b), etc.

In still further aspects, the present disclosure contemplates a method of determining a therapeutic dosage range of an IL-10 agent for subcutaneous administration to a subject for the treatment of a cholesterol-related disease, disorder or condition, comprising a) determining a minimum dose of the IL-10 agent, wherein the minimum dose is an amount of the IL-10 agent administered subcutaneously to the subject that provides an approximately 10% reduction in the total serum cholesterol concentration at least 24 hours after the dose compared to a baseline concentration of total serum cholesterol in the subject, and b) determining a maximum dose of the IL-10 agent, wherein the maximum dose is the highest amount of the IL-10 agent that can be administered subcutaneously to the subject without causing an unacceptable indicia of an IL-10 agent—associated toxicity at a steady state serum level of the IL-10 agent, wherein the therapeutic dosage range of the IL-10 agent is the minimum dose determined in step a) and the maximum dose determined in step b).

A particular embodiment of the present disclosure is drawn to a method of determining whether a treatment regimen of an IL-10 agent administered subcutaneously to a subject having a cholesterol-related disease, disorder or condition is suboptimal, comprising a) determining the total serum cholesterol concentration in the subject at one or more times subsequent to achieving an IL-10 steady state concentration, and b) comparing the total serum cholesterol concentration determined in step a) with a baseline concentration in the subject determined prior to initiation of the treatment regimen, wherein a suboptimal dose is an amount of the IL-10 agent insufficient to achieve at least a 10% reduction in the total serum cholesterol in the subject.

The skilled artisan will be able to optimize the amount of the IL-10 agent for the treatment of a particular subject by extrapolating from the data resulting from the methods described herein. In certain instances, the skilled artisan will arrive at the optimal amount of the IL-10 agent by an iterative process comprising conducting a described method multiple times.

A number of different factors, including many of which are subject-specific, can influence the desired reduction in total serum cholesterol. A clinician skilled in the art will be able to identify an appropriate amount of reduction under the particular circumstances at hand. For example, in some embodiments, a reduction in total serum cholesterol of at least 10% is appropriate, while in other embodiments the reduction in total serum cholesterol is at least 12%, at least 15% or more.

The present disclosure contemplates the use of the methods in embodiments wherein the cholesterol-related disease, disorder or condition is a cardiovascular disorder. In some instances, the cardiovascular disorder is atherosclerosis, a cardiomyopathy, or a hypertensive disorder. Further embodiments are contemplated wherein the subject has hypercholesterolemia or a lipidemia. In other embodiments, the cholesterol-related disease, disorder or condition is a thrombotic disorder, which can cause stroke or myocardial infarction in some subjects. In other embodiments contemplated herein, the cholesterol-related disease, disorder or condition is an inflammatory disorder (e.g., a vasculitis).

The present disclosure contemplates embodiments wherein the IL-10 agent is mature human IL-10, or is a variant of mature human IL-10 wherein the variant exhibits activity comparable to the activity of mature human IL-10. In particular embodiments, the IL-10 agent comprises at least one modification to form a modified IL-10 agent, wherein the modification does not alter the amino acid sequence of the IL-10 agent. The modification is site-specific and/or comprises a linker in certain embodiments of the present disclosure.

An example of such a modification is a pegylated IL-10 agent. In particular embodiments, the pegylated IL-10 agent comprises at least one PEG molecule covalently attached to at least one amino acid residue of at least one subunit of IL-10. The pegylated IL-10 agent comprises a mixture of mono-pegylated and di-pegylated IL-10 in other embodiments. The present disclosure is not limited by the size of the PEG component. The PEG component of the PEG-IL-10 agent may have a molecular mass of less than 5 kDa, a molecular mass of about 5 kDa, a molecular mass of greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa or from about 10 kDa to about 30 kDa.

The present disclosure contemplates methods of treating or preventing a cholesterol-related disease, disorder or condition in a subject, comprising administering parenterally to the subject an amount of an IL-10 agent determined by any of the aforementioned methods. In some of these methods, the amount is sufficient to maintain a mean IL-10 serum trough concentration (e.g., from about 0.1 ng/mL to about 5.0 ng/mL) over a particular period of time (e.g., the mean IL-10 serum trough concentration is maintained for at least 95% over a period of 1 week). A comprehensive discussion of exemplary serum trough concentrations, periods of time, etc. appears hereafter.

In some embodiments of the present disclosure, the mean IL-10 serum trough concentration is in the range of from 1.0 pg/mL to 100 pg/mL; from 0.1 ng/mL to 1.0 ng/mL; from 1.0 ng/mL to 10 ng/mL; from 0.5 ng/mL to 5.0 ng/mL; from 0.75 ng/mL to 1.25 ng/mL or from 0.9 ng/mL to 1.1 ng/mL. In particular embodiments of the present disclosure, the mean IL-10 serum trough concentration is at least 0.1 ng/mL, at least 0.5 ng/mL, at least 1.0 ng/mL, 1.25 ng/mL, at least 1.5 ng/mL, at least 1.6 ng/mL, at least 1.7 ng/mL, at least 1.8 ng/mL, at least 1.85 ng/mL, at least 1.9 ng/mL, at least 1.95 ng/mL, at least 1.97 ng/mL, and least 1.98 ng/mL, at least 1.99 ng/mL, at least 2.0 ng/mL or greater than 2 ng/mL.

The present disclosure is not limited to any specific period of time(s) in which the mean IL-10 serum trough concentration is maintained. In some embodiments, the period of time is at least 12 hours, at least 24 hours, at least 48 hours, at least 72 hours, at least 1 week, at least 2 weeks, at least 3 weeks, at least 1 month, at least 6 weeks, at least 2 months, at least 3 months, at least 6 months, at least 9 months, or greater than 12 months.

In particular embodiments of the present disclosure, the mean IL-10 serum trough concentration is maintained for at least 85% of the period of time, at least 90%, at least 96%, at least 98%, at least 99% or 100% of the period of time.

It is envisaged that a dosing regimen sufficient to maintain a desired steady state serum trough concentration (e.g., 1 ng/mL) may result in an initial serum trough concentration that is higher than the desired steady state serum trough concentration. Because of the pharmacodynamic and pharmacokinetic characteristics of IL-10 in a mammalian subject, an initial trough concentration (achieved, for example, through the administration of one or more loading doses followed by a series of maintenance doses) gradually but continually decreases over a period of time even when the dosing parameters (amount and frequency) are kept constant. After that period to time, the gradual but continual decrease ends and a steady state serum trough concentration is maintained.

By way of example, parenteral administration (e.g., SC and IV) of ˜0.1 mg/kg/day of an IL-10 agent (e.g., mIL-10) to a mouse (e.g., a C57BL/6 mouse) is required to maintain a steady state serum trough concentration of 2.0 ng/mL. However, that steady state serum trough concentration may not be achieved until approximately 30 days after initiation of dosing at 0.1 mg/kg/day (and also after any loading dose(s)). Rather, after an initial serum trough concentration has been achieved (e.g., 2.5 ng/mL), that concentration gradually but continually decreases over the course of, for example, the approximately 30-day period, after which time the desired steady state serum trough concentration (2.0 ng/mL) is maintained. One of skill in the art will be able to determine the dose needed to maintain the desired steady state trough concentration using, for example, ADME and patient-specific parameters.

In further embodiments of the present disclosure, an IL-10 agent described herein is administered in combination with at least one additional prophylactic and/or therapeutic agent. In some embodiments, the prophylactic and/or therapeutic agent is a cholesterol homeostasis agent (e.g., a statin, a bile acid resin, ezetimibe, a fibric acid, a niacin, or a PCSK9 inhibitor). In other embodiments, the prophylactic and/or therapeutic agent is an anti-diabetic agent or an anti-obesity agent, while in still other embodiments the prophylactic or therapeutic agent is an immune agent or an anti-inflammatory agent. Additional examples of such prophylactic and/or therapeutic agents are described further below.

Also contemplated herein are pharmaceutical compositions comprising a pharmaceutically effective amount of an IL-10 agent, as determined in any of the aforementioned methods, and a pharmaceutically acceptable diluent, carrier or excipient (e.g., an isotonic injection solution). In particular embodiments, the composition is suitable for human administration. The pharmaceutical compositions can further comprise at least one additional prophylactic and/or therapeutic agent (e.g., a cholesterol homeostasis agent, an anti-diabetic agent, an anti-obesity agent, an immune agent, or an anti-inflammatory agent).

Certain embodiments of the present disclosure contemplate a sterile container that contains one of the above-mentioned pharmaceutical compositions and optionally one or more additional components. By way of example, but not limitation, the sterile container may be a syringe. In still further embodiments, the sterile container is one component of a kit; the kit may also contain, for example, a second sterile container that comprises at least one prophylactic or therapeutic agent, examples of which are set forth herein.

### BRIEF DESCRIPTION OF THE DRAWINGS

FIG. 1 depicts the amino acid sequences of human and mouse IL-10.

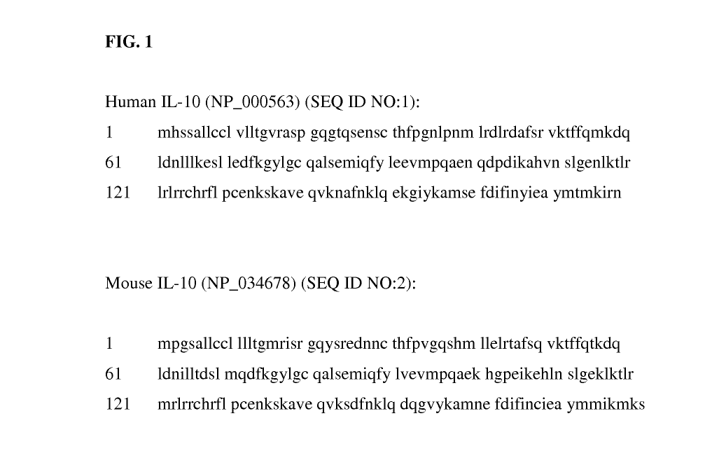

FIG. 2A depicts the effect of SC administration of a single ascending dose of rhIL-10 (placebo, 1.0 μg/kg, 2.5 μg/kg, 5.0 μg/kg, 10 μg/kg, 25 μg/kg and 50 μg/kg) on total serum cholesterol in normal, healthy human males. Data are normalized to the starting values; n≧6.

FIG. 2B depicts the effect of IV administration of a single ascending dose of rhIL-10 (placebo, 1.0 μg/kg, 5.0 μg/kg, 10 μg/kg, 25 μg/kg 50 μg/kg and 100 μg/kg) on total serum cholesterol in normal, healthy human males. Data are normalized to the starting values; n≧6.

FIG. 3 depicts the effect of several subcutaneous IL-10 dosing regimens on reduction of total serum cholesterol in human subjects having liver fibrosis resulting from chronic hepatitis C (n=19-13 males/group).

### DETAILED DESCRIPTION

Before the present disclosure is further described, it is to be understood that the disclosure is not limited to the particular embodiments set forth herein, and it is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. Unless defined otherwise, all technical and scientific terms used herein have the same meaning as commonly understood by one of ordinary skill in the art to which this invention belongs.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology such as “solely,” “only” and the like in connection with the recitation of claim elements, or use of a “negative” limitation.

The publications discussed herein are provided solely for their disclosure prior to the filing date of the present application. Further, the dates of publication provided may be different from the actual publication dates, which may need to be independently confirmed.

### Overview

The present disclosure pertains to methods and compositions comprising IL-10 agents (e.g., PEG-IL-10) and their association with the treatment and/or prevention of various diseases, disorders and conditions, and/or the symptoms thereof. More particularly, the present disclosure relates to, for example, methods for determining amounts of an IL-10 agent (e.g., PEG-IL-10) for subcutaneous administration that are therapeutically useful for the treatment and/or prevention of a cholesterol-related disease, disorder or condition in a subject (e.g., a human); dosing regimens comprising the administration of such amounts; and pharmaceutical compositions comprising the amounts of the IL-10 agents.

Certain embodiments are directed to means for establishing a therapeutic range of an IL-10 agent in a subject. In some embodiments, particular parameters (e.g., hematological parameters) are monitored to provide an indication of the upper limit of the therapeutic range such that any untoward adverse effects will be avoided. A primary basis for the foregoing is the inventors' unexpected finding that SC administration of a particular dose of IL-10 is pharmacodynamically more active than a much larger dose administered IV, even when similar serum trough concentrations are compared.

It should be noted that any reference to “human” in connection with the polypeptides and nucleic acid molecules of the present disclosure is not meant to be limiting with respect to the manner in which the polypeptide or nucleic acid is obtained or the source, but rather is only with reference to the sequence as it may correspond to a sequence of a naturally occurring human polypeptide or nucleic acid molecule. In addition to the human polypeptides and the nucleic acid molecules which encode them, the present disclosure contemplates IL-10—related polypeptides and corresponding nucleic acid molecules from other species.

### DEFINITIONS

Unless otherwise indicated, the following terms are intended to have the meaning set forth below. Other terms are defined elsewhere throughout the specification.

The terms “subject” or “patient” are used interchangeably to refer to a human or a non-human animal (e.g., a mammal).

The terms “administration”, “administer” and the like, as they apply to, for example, a subject, cell, tissue, organ, or biological fluid, refer to contact of, for example, IL-10 or PEG-IL-10), a nucleic acid (e.g., a nucleic acid encoding native human IL-10); a pharmaceutical composition comprising the foregoing, or a diagnostic agent to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

The terms “treat”, “treating”, treatment” and the like refer to a course of action (such as administering IL-10 or a pharmaceutical composition comprising IL-10) initiated after a disease, disorder or condition, or a symptom thereof, has been diagnosed, observed, and the like so as to eliminate, reduce, suppress, mitigate, or ameliorate, either temporarily or permanently, at least one of the underlying causes of a disease, disorder, or condition afflicting a subject, or at least one of the symptoms associated with a disease, disorder, condition afflicting a subject. Thus, treatment includes inhibiting (e.g., arresting the development or further development of the disease, disorder or condition or clinical symptoms association therewith) an active disease. The terms may also be used in other contexts, such as situations where IL-10 or PEG-IL-10 contacts an IL-10 receptor in, for example, the fluid phase or colloidal phase.

The term “in need of treatment” as used herein refers to a judgment made by a physician or other caregiver that a subject requires or will benefit from treatment. This judgment is made based on a variety of factors that are in the realm of the physician's or caregiver's expertise.

The terms “prevent”, “preventing”, “prevention” and the like refer to a course of action (such as administering IL-10 or a pharmaceutical composition comprising IL-10) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state.

The term “in need of prevention” as used herein refers to a judgment made by a physician or other caregiver that a subject requires or will benefit from preventative care. This judgment is made based on a variety of factors that are in the realm of a physician's or caregiver's expertise.

The phrases “therapeutically effective amount”, “pharmaceutically effective amount”, and the like, refer to the administration of an agent to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like. By way of example, measurement of the amount of inflammatory cytokines produced following administration may be indicative of whether a therapeutically effective amount has been used.

The phrase “in a sufficient amount to effect a change” means that there is a detectable difference between a level of an indicator measured before (e.g., a baseline level) and after administration of a particular therapy. Indicators include any objective parameter (e.g., serum concentration of IL-10) or subjective parameter (e.g., a subject's feeling of well-being).

The term “small molecules” refers to chemical compounds having a molecular weight that is less than about 10 kDa, less than about 2 kDa, or less than about lkDa. Small molecules include, but are not limited to, inorganic molecules, organic molecules, organic molecules containing an inorganic component, molecules comprising a radioactive atom, and synthetic molecules. Therapeutically, a small molecule may be more permeable to cells, less susceptible to degradation, and less likely to elicit an immune response than large molecules (e.g., compositions comprising an antibody).

The term “ligand” refers to, for example, a peptide, polypeptide, membrane-associated or membrane-bound molecule, or complex thereof, that can act as an agonist or antagonist of a receptor. “Ligand” encompasses natural and synthetic ligands, e.g., cytokines, cytokine variants, analogs, muteins, and binding compositions derived from antibodies. “Ligand” also encompasses small molecules, e.g., peptide mimetics of cytokines and peptide mimetics of antibodies. The term also encompasses an agent that is neither an agonist nor antagonist, but that can bind to a receptor without significantly influencing its biological properties, e.g., signaling or adhesion. Moreover, the term includes a membrane-bound ligand that has been changed, e.g., by chemical or recombinant methods, to a soluble version of the membrane-bound ligand. A ligand or receptor may be entirely intracellular, that is, it may reside in the cytosol, nucleus, or some other intracellular compartment. The complex of a ligand and receptor is termed a “ligand-receptor complex.”

The terms “inhibitors” and “antagonists”, or “activators” and “agonists” refer to inhibitory or activating molecules, respectively, Inhibitors are molecules that decrease, block, prevent, delay activation, inactivate, desensitize, or down-regulate, e.g., a gene, protein, ligand, receptor, or cell. Activators are molecules that increase, activate, facilitate, enhance activation, sensitize, or up-regulate, e.g., a gene, protein, ligand, receptor, cofactor, or cell. An inhibitor may also be defined as a molecule that reduces, blocks, or inactivates a constitutive activity. An “agonist” is a molecule that interacts with a target to cause or promote an increase in the activation of the target. An “antagonist” is a molecule that opposes the action(s) of an agonist. An antagonist prevents, reduces, inhibits, or neutralizes the activity of an agonist, and an antagonist can also prevent, inhibit, or reduce constitutive activity of a target, e.g., a target receptor, even where there is no identified agonist.

The terms “modulate”, “modulation” and the like refer to the ability of a molecule (e.g., an activator or an inhibitor) to increase or decrease the function or activity of IL-10 agents (or the nucleic acid molecules encoding them), either directly or indirectly; or to enhance the ability of a molecule to produce an effect comparable to that of an IL-10 agent. The term “modulator” is meant to refer broadly to molecules that can effect the activities described above. By way of example, a modulator of, e.g., a gene, receptor, ligand, cofactor, or cell, is a molecule that alters an activity of the gene, receptor, ligand, cofactor, or cell, where activity can be activated, inhibited, or altered in its regulatory properties. A modulator may act alone, or it may use a cofactor, e.g., a protein, metal ion, or small molecule. The term “modulator” includes agents that operate through the same or similar mechanism of action as IL-10 (i.e., agents that modulate the same signaling pathway as IL-10 in a manner analogous thereto) and are capable of eliciting a biological response comparable to (or greater than) that of IL-10.

Examples of modulators include small molecule compounds and other bioorganic molecules. Numerous libraries of small molecule compounds (e.g., combinatorial libraries) are commercially available and can serve as a starting point for identifying a modulator. The skilled artisan is able to develop one or more assays (e.g., biochemical or cell-based assays) in which such compound libraries can be screened in order to identify one or more compounds having the desired properties; thereafter, the skilled medicinal chemist is able to optimize such one or more compounds by, for example, synthesizing and evaluating analogs and derivatives thereof. Synthetic and/or molecular modeling studies can also be utilized in the identification of an Activator.

The “activity” of a molecule may describe or refer to the binding of the molecule to a ligand or to a receptor; to catalytic activity; to the ability to stimulate gene expression or cell signaling, differentiation, or maturation; to antigenic activity; to the modulation of activities of other molecules; and the like. The term may also refer to activity in modulating or maintaining cell-to-cell interactions (e.g., adhesion), or activity in maintaining a structure of a cell (e.g., a cell membrane). “Activity” can also mean specific activity, e.g., [catalytic activity]/[mg protein], or [immunological activity]/[mg protein], concentration in a biological compartment, or the like. The term “proliferative activity” encompasses an activity that promotes, that is necessary for, or that is specifically associated with, for example, normal cell division, as well as cancer, tumors, dysplasia, cell transformation, metastasis, and angiogenesis.

As used herein, “comparable”, “comparable activity”, “activity comparable to”, “comparable effect”, “effect comparable to”, and the like are relative terms that can be viewed quantitatively and/or qualitatively. The meaning of the terms is frequently dependent on the context in which they are used. By way of example, two agents that both activate a receptor can be viewed as having a comparable effect from a qualitative perspective, but the two agents can be viewed as lacking a comparable effect from a quantitative perspective if one agent is only able to achieve 20% of the activity of the other agent as determined in an art-accepted assay (e.g., a dose-response assay) or in an art-accepted animal model. When comparing one result to another result (e.g., one result to a reference standard), “comparable” frequently means that one result deviates from a reference standard by less than 35%, by less than 30%, by less than 25%, by less than 20%, by less than 15%, by less than 10%, by less than 7%, by less than 5%, by less than 4%, by less than 3%, by less than 2%, or by less than 1%. In particular embodiments, one result is comparable to a reference standard if it deviates by less than 15%, by less than 10%, or by less than 5% from the reference standard. By way of example, but not limitation, the activity or effect may refer to efficacy, stability, solubility, or immunogenicity.

The term “response,” for example, of a cell, tissue, organ, or organism, encompasses a change in biochemical or physiological behavior, e.g., concentration, density, adhesion, or migration within a biological compartment, rate of gene expression, or state of differentiation, where the change is correlated with activation, stimulation, or treatment, or with internal mechanisms such as genetic programming. In certain contexts, the terms “activation”, “stimulation”, and the like refer to cell activation as regulated by internal mechanisms, as well as by external or environmental factors; whereas the terms “inhibition”, “down-regulation” and the like refer to the opposite effects.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence; fusion proteins with heterologous and homologous leader sequences; fusion proteins with or without N-terminus methionine residues; fusion proteins with immunologically tagged proteins; and the like.

It will be appreciated that throughout this disclosure reference is made to amino acids according to the single letter or three letter codes. For the reader's convenience, the single and three letter amino acid codes are provided below:

| | | | | | |
|---|---|---|---|---|---|
| G | Glycine | Gly | P | Proline | Pro |
| A | Alanine | Ala | V | Valine | Val |
| L | Leucine | Leu | I | Isoleucine | Ile |
| M | Methionine | Met | C | Cysteine | Cys |
| F | Phenylalanine | Phe | Y | Tyrosine | Tyr |
| W | Tryptophan | Trp | H | Histidine | His |
| K | Lysine | Lys | R | Arginine | Arg |
| Q | Glutamine | Gln | N | Asparagine | Asn |
| E | Glutamic Acid | Glu | D | Aspartic Acid | Asp |
| S | Serine | Ser | T | Threonine | Thr |

As used herein, the term “variant” encompasses naturally-occurring variants and non-naturally-occurring variants. Naturally-occurring variants include homologs (polypeptides and nucleic acids that differ in amino acid or nucleotide sequence, respectively, from one species to another), and allelic variants (polypeptides and nucleic acids that differ in amino acid or nucleotide sequence, respectively, from one individual to another within a species). Non-naturally-occurring variants include polypeptides and nucleic acids that comprise a change in amino acid or nucleotide sequence, respectively, where the change in sequence is artificially introduced (e.g., muteins); for example, the change is generated in the laboratory by human intervention (“hand of man”). Thus, herein a “mutein” refers broadly to mutated recombinant proteins that usually carry single or multiple amino acid substitutions and are frequently derived from cloned genes that have been subjected to site-directed or random mutagenesis, or from completely synthetic genes.

The terms “DNA”, “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the like.

As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” or “immediately C-terminal” refers to a position of a first amino acid residue relative to a second amino acid residue where the first and second amino acid residues are covalently bound to provide a contiguous amino acid sequence.

“Derived from”, in the context of an amino acid sequence or polynucleotide sequence (e.g., an amino acid sequence “derived from” an IL-10 polypeptide), is meant to indicate that the polypeptide or nucleic acid has a sequence that is based on that of a reference polypeptide or nucleic acid (e.g., a naturally occurring IL-10 polypeptide or an IL-10-encoding nucleic acid), and is not meant to be limiting as to the source or method in which the protein or nucleic acid is made. By way of example, the term “derived from” includes homologs or variants of reference amino acid or DNA sequences.

In the context of a polypeptide, the term “isolated” refers to a polypeptide of interest that, if naturally occurring, is in an environment different from that in which it may naturally occur. “Isolated” is meant to include polypeptides that are within samples that are substantially enriched for the polypeptide of interest and/or in which the polypeptide of interest is partially or substantially purified. Where the polypeptide is not naturally occurring, “isolated” indicates that the polypeptide has been separated from an environment in which it was made by either synthetic or recombinant means.

“Enriched” means that a sample is non-naturally manipulated (e.g., by a scientist) so that a polypeptide of interest is present in a) a greater concentration (e.g., at least 3-fold greater, at least 4-fold greater, at least 8-fold greater, at least 64-fold greater, or more) than the concentration of the polypeptide in the starting sample, such as a biological sample (e.g., a sample in which the polypeptide naturally occurs or in which it is present after administration), or b) a concentration greater than the environment in which the polypeptide was made (e.g., as in a bacterial cell).

“Substantially pure” indicates that a component (e.g., a polypeptide) makes up greater than about 50% of the total content of the composition, and typically greater than about 60% of the total polypeptide content. More typically, “substantially pure” refers to compositions in which at least 75%, at least 85%, at least 90% or more of the total composition is the component of interest. In some cases, the polypeptide will make up greater than about 90%, or greater than about 95% of the total content of the composition.

The terms “specifically binds” or “selectively binds”, when referring to a ligand/receptor, antibody/antigen, or other binding pair, indicates a binding reaction which is determinative of the presence of the protein in a heterogeneous population of proteins and other biologics. By way of example, under designated conditions, a specified ligand binds to a particular receptor and does not bind in a significant amount to other proteins present in the sample. The antibody, or binding composition derived from the antigen-binding site of an antibody, of the contemplated method binds to its antigen, or a variant or mutein thereof, with an affinity that is at least two-fold greater, at least ten times greater, at least twenty times greater, or at least one-hundred times greater than the affinity with any other antibody, or binding composition derived therefrom. In a particular embodiment, the antibody will have an affinity that is greater than about 10⁹ liters/mol, as determined by, e.g., Scatchard analysis (Munsen, et al. 1980 Analyt. Biochem. 107:220-239).

As used herein, the phrase “body weight disorder” refers to conditions associated with excessive body weight and/or enhanced appetite. Various parameters are used to determine whether a subject is overweight compared to a reference healthy individual, including the subject's age, height, sex and health status. For example, a subject may be considered overweight or obese by assessment of the subject's Body Mass Index (BMI), which is calculated by dividing a subject's weight in kilograms by the subject's height in meters squared. An adult having a BMI in the range of ˜18.5 to ˜24.9 kg/m² is considered to have a normal weight; an adult having a BMI between ˜25 and ˜29.9 kg/m² may be considered overweight (pre-obese); and an adult having a BMI of ˜30 kg/m² or higher may be considered obese. Enhanced appetite frequently contributes to excessive body weight. There are several conditions associated with enhanced appetite, including, for example, night eating syndrome, which is characterized by morning anorexia and evening polyphagia often associated with insomnia, but which may be related to injury to the hypothalamus.

The terms “antibodies” (Abs) and “immunoglobulins” (Igs) refer to glycoproteins having the same structural characteristics. While antibodies exhibit binding specificity to a specific antigen, immunoglobulins include both antibodies and other antibody-like molecules which lack antigen specificity.

The term “monoclonal antibody” refers to an antibody obtained from a population of substantially homogeneous antibodies, that is, the individual antibodies comprising the population are identical except for possible naturally occurring mutations that may be present in minor amounts. Monoclonal antibodies are highly specific, being directed against a single antigenic site. In contrast to polyclonal antibody preparations, which can include different antibodies directed against different determinants (epitopes), each monoclonal antibody is directed against a single determinant on the antigen.

In the context of an antibody, the term “isolated” refers to an antibody that has been separated and/or recovered from contaminant components of its natural environment; such contaminant components include materials which might interfere with diagnostic or therapeutic uses for the antibody, and may include enzymes, hormones, and other proteinaceous or nonproteinaceous solutes.

### IL-10 and PEG-IL-10

IL-10, also known as human cytokine synthesis inhibitory factor (CSIF), is classified as a type(class)-2 cytokine, a set of cytokines that includes IL-19, IL-20, IL-22, IL-24 (Mda-7), and IL-26, interferons (IFN-α, -β, -γ, -δ, -ε, -κ, -Ω, and -τ) and interferon-like molecules (limitin, IL-28A, IL-28B, and IL-29). IL-10 is a cytokine with pleiotropic effects in immunoregulation and inflammation. It is produced by mast cells, counteracting the inflammatory effects that these cells have at the site of an allergic reaction. While it is capable of inhibiting the synthesis of pro-inflammatory cytokines such as IFN-γ, IL-2, IL-3, TNFα and GM-CSF, IL-10 is also stimulatory towards certain T-cells and mast cells and stimulates B-cell maturation, proliferation and antibody production. IL-10 can block NF-κB activity and is involved in the regulation of the JAK-STAT signaling pathway. It also induces the cytotoxic activity of CD8+ T-cells and the antibody production of B-cells, and it suppresses macrophage activity and tumor-promoting inflammation. The regulation of CD8+ T-cells is dose-dependent, wherein higher doses induce stronger cytotoxic responses.

Human IL-10 is a homodimer with a molecular mass of 37 kDa, wherein each 18.5 kDa monomer comprises 178 amino acids, the first 18 of which comprise a signal peptide, and two cysteine residues that form two intramolecular disulfide bonds. The IL-10 dimer becomes biologically inactive upon disruption of the non-covalent interactions between the two monomeric subunits.

The present disclosure contemplates human IL-10 and murine IL-10, which exhibit 80% homology, and use thereof. In addition, the scope of the present disclosure includes IL-10 orthologs, and modified forms thereof, from other mammalian species, including rat (accession NP 036986.2; GI 148747382); cow (accession NP_776513.1; GI 41386772); sheep (accession NP_001009327.1; GI 57164347); dog (accession ABY86619.1; GI 166244598); and rabbit (accession AAC23839.1; GI 3242896).

As alluded to above, the terms “IL-10”, “IL-10 polypeptide(s), “IL-10 agent(s)” and the like are intended to be broadly construed and include, for example, human and non-human IL-10—related polypeptides, including homologs, variants (including muteins), and fragments thereof, as well as IL-10 polypeptides having, for example, a leader sequence (e.g., the signal peptide), and modified versions of the foregoing. In further particular embodiments, IL-10, IL-10 polypeptide(s), and IL-10 agent(s) are agonists.

The IL-10 receptor, a type II cytokine receptor, consists of alpha and beta subunits, which are also referred to as R1 and R2, respectively. Receptor activation requires binding to both alpha and beta; one homodimer of an IL-10 polypeptide binds to alpha and the other homodimer of the same IL-10 polypeptide binds to beta.

The utility of recombinant human IL-10 is frequently limited by its relatively short serum half-life, which may be due to, for example, renal clearance, proteolytic degradation and monomerization in the blood stream. As a result, various approaches have been explored to improve the pharmacokinetic profile of IL-10 without disrupting its dimeric structure and thus adversely affecting its activity. Pegylation of IL-10 results in, for example, improvement of certain pharmacokinetic parameters (e.g., serum half-life) and/or enhancement of activity.

As used herein, the terms “pegylated IL-10” and PEG-IL-10” refer to an IL-10 molecule having one or more polyethylene glycol molecules covalently attached to at least one amino acid residue of the IL-10 protein, generally via a linker, such that the attachment is stable. The terms “monopegylated IL-10” and “mono-PEG-IL-10” indicate that one polyethylene glycol molecule is covalently attached to a single amino acid residue on one subunit of the IL-10 dimer, generally via a linker. In certain embodiments, the PEG-IL-10 used in the present disclosure is a mono-PEG-IL-10 in which one to nine PEG molecules are covalently attached via a linker to the alpha amino group of the amino acid residue at the N-terminus of one subunit of the IL-10 dimer. Monopegylation on one IL-10 subunit generally results in a non-homogeneous mixture of non-pegylated, monopegylated and dipegylated IL-10 due to subunit shuffling. Moreover, allowing a pegylation reaction to proceed to completion will generally result in non-specific and multi-pegylated IL-10, thus reducing its bioactivity. Thus, particular embodiments of the present disclosure comprise the administration of a mixture of mono- and di-pegylated IL-10 produced by the methods described herein.

In particular embodiments, the average molecular weight of the PEG moiety is between about 5 kDa and about 50 kDa. Additional aspects and characteristics of pegylation are described herein. Although the method or site of PEG attachment to IL-10 is not critical, in certain embodiments the pegylation does not alter, or only minimally alters, the activity of the IL-10 agent. In certain embodiments, the increase in half-life is greater than any decrease in biological activity. The biological activity of PEG-IL-10 is typically measured by assessing the levels of inflammatory cytokines (e.g., TNF-α or IFN-γ) in the serum of subjects challenged with a bacterial antigen (lipopolysaccharide (LPS)) and treated with PEG-IL-10, as described in U.S. Pat. No. 7,052,686.

IL-10 variants can be prepared with various objectives in mind, including increasing serum half-life, reducing an immune response against the IL-10, facilitating purification or preparation, decreasing conversion of IL-10 into its monomeric subunits, improving therapeutic efficacy, and lessening the severity or occurrence of side effects during therapeutic use. The amino acid sequence variants are usually predetermined variants not found in nature, although some may be post-translational variants, e.g., glycosylated variants. Any variant of IL-10 can be used provided it retains a suitable level of IL-10 activity.

The phrase “conservative amino acid substitution” refers to substitutions that preserve the activity of the protein by replacing an amino acid(s) in the protein with an amino acid with a side chain of similar acidity, basicity, charge, polarity, or size of the side chain. Conservative amino acid substitutions generally entail substitution of amino acid residues within the following groups: 1) L, I, M, V, F; 2) R, K; 3) F, Y, H, W, R; 4) G, A, T, S; 5) Q, N; and 6) D, E. Guidance for substitutions, insertions, or deletions may be based on alignments of amino acid sequences of different variant proteins or proteins from different species. Thus, in addition to any naturally-occurring IL-10 polypeptide, the present disclosure contemplates having 1, 2, 3, 4, 5, 6, 7, 8, 9, or 10 usually no more than 20, 10, or 5 amino acid substitutions, where the substitution is usually a conservative amino acid substitution.

The present disclosure also contemplates active fragments (e.g., subsequences) of mature IL-10 containing contiguous amino acid residues derived from the mature IL-10. The length of contiguous amino acid residues of a peptide or a polypeptide subsequence varies depending on the specific naturally-occurring amino acid sequence from which the subsequence is derived. In general, peptides and polypeptides may be from about 20 amino acids to about 40 amino acids, from about 40 amino acids to about 60 amino acids, from about 60 amino acids to about 80 amino acids, from about 80 amino acids to about 100 amino acids, from about 100 amino acids to about 120 amino acids, from about 120 amino acids to about 140 amino acids, from about 140 amino acids to about 150 amino acids, from about 150 amino acids to about 155 amino acids, and from about 155 amino acids up to the full-length peptide or polypeptide.

Additionally, IL-10 polypeptides can have a defined sequence identity compared to a reference sequence over a defined length of contiguous amino acids (e.g., a “comparison window”). Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Madison, Wis.), or by manual alignment and visual inspection (see, e.g., Current Protocols in Molecular Biology (Ausubel et al., eds. 1995 supplement)).

As an example, a suitable IL-10 polypeptide can comprise an amino acid sequence having at least about 75%, at least about 80%, at least about 85%, at least about 90%, at least about 95%, at least about 98%, or at least about 99%, amino acid sequence identity to a contiguous stretch of from about 20 amino acids to about 40 amino acids, from about 40 amino acids to about 60 amino acids, from about 60 amino acids to about 80 amino acids, from about 80 amino acids to about 100 amino acids, from about 100 amino acids to about 120 amino acids, from about 120 amino acids to about 140 amino acids, from about 140 amino acids to about 150 amino acids, from about 150 amino acids to about 155 amino acids, or from about 155 amino acids up to the full-length peptide or polypeptide.

As discussed further below, the IL-10 polypeptides may be isolated from a natural source (e.g., an environment other than its naturally-occurring environment) and may also be recombinantly made (e.g., in a genetically modified host cell such as bacteria, yeast, Pichia, insect cells, and the like), where the genetically modified host cell is modified with a nucleic acid comprising a nucleotide sequence encoding the polypeptide. The IL-10 polypeptides may also be synthetically produced (e.g., by cell-free chemical synthesis).

Nucleic acid molecules encoding the IL-10 agents are contemplated by the present disclosure, including their naturally-occurring and non-naturally occurring isoforms, allelic variants and splice variants. The present disclosure also encompasses nucleic acid sequences that vary in one or more bases from a naturally-occurring DNA sequence but still translate into an amino acid sequence that corresponds to an IL-10 polypeptide due to degeneracy of the genetic code.

### IL-10—Specific Dosing and Serum Concentration Parameters

IL-10 Dosing:

The present disclosure contemplates the use of the agents described herein, and compositions thereof, to treat and/or prevent various metabolic-related diseases (e.g., hypercholesterolemia), disorders and conditions, and/or the symptoms thereof, via the subcutaneous administration of an IL-10 agent (e.g., PEG-IL-10). In certain aspects of the present disclosure, such treatment or prevention is effected by identifying particular dosing parameters, while in other aspects it is effected by utilizing particular dosing parameters. Other embodiments include therapeutically effective compositions. The present disclosure is based on the findings that there is an optimal mean IL-10 serum trough concentration range and an optimal dosing range that achieves therapeutically relevant reduction of serum cholesterol and avoids severe toxicity resulting from higher IL-10 serum concentrations.

More particularly, the present disclosure relates to, for example, methods for determining amounts of an IL-10 agent (e.g., PEG-IL-10) for subcutaneous administration that are therapeutically useful for the treatment and/or prevention of a cholesterol-related disease, disorder or condition in a subject (e.g., a human); dosing regimens comprising the administration of such amounts; and pharmaceutical compositions comprising the amounts of the IL-10 agents. Certain embodiments are directed to means for establishing a therapeutic range of an IL-10 agent in a subject. In some embodiments, particular parameters (e.g., hematological parameters) are monitored to provide an indication of the upper limit of the therapeutic range such that any untoward adverse effects will be avoided.

As described herein, one aspect of the present disclosure is the identification of a therapeutically effective amount of an IL-10 agent for subcutaneous administration to a subject to effect the desired reduction of total serum cholesterol (e.g., at least 10% compared to baseline, at least 11%, at least 12%, or more) at a defined time post-administration (e.g., 24 or 48 hours), and the absence of an unacceptable indicia of an IL-10 agent—associated toxicity in the subject. In this context, the “floor” of the therapeutic range (or therapeutic index) is the minimum IL-10 serum concentration (at steady state) sufficient to maintain the desired therapeutic effect (e.g., at least a 10% reduction in total serum cholesterol measured 48 hours after dosing). The “ceiling” of the therapeutic range is the maximum IL-10 serum concentration (at steady state) above which undesired effects are observed. As used herein, the phrase “absence of an unacceptable indicia of an IL-10 agent—associated toxicity in the subject” refers to the ceiling of the therapeutic range.

Untoward Effects of IL-10:

The present disclosure contemplates the use of any subjective (e.g., a subject's feeling of well-being) or objective (e.g., an unacceptable reduction in hematocrit) parameter as a basis for determining whether a subject is experiencing an unacceptable indicia of an IL-10 agent—associated toxicity, which would necessitate (or at least suggest) a reduction in the amount of the subcutaneously administered IL-10 agent. It must be emphasized that the aforementioned parameters are subject-specific, as is the dosing regimen(s) that obviates such toxicity.

Anemia is a parameter that can generally be used as a basis for determining whether an unacceptable toxicity is present in a subject. Anemia is commonly associated with many diverse systemic inflammatory conditions, including infection, rheumatologic disorders, and cancer. The cause of anemia of chronic disease is multifactorial and includes both inhibition of RBC production and decreased RBC lifespan. The fact that cytokines are thought to play an important role in many of the diseases associated with anemia, and that levels of many of these cytokines often correlate with the degree of anemia, suggest an association between them.

IL-10 and other inflammatory cytokines have been shown to play a role in many of the underlying diseases associated with anemia of chronic disease. It is postulated that there is a mechanistic link between IL-10 and the pathophysiology of anemia, including decreased erythrocyte survival. Moreover, studies have shown that IL-10 may directly alter certain aspects of RBC hemostasis, including the kinetics and number of RBCs (Moldawer et al., FASEB J 3:1637 (1989).

In clinical trials, IL-10 led to anemia in patients with Crohn's disease. IL-10 has also been shown to increase ferritin levels, resulting in decreased iron availability for RBC precursors and concomitant reduction in RBC number, and IL-10 also appears to increase ferritin translation (Tilg et al., J Immunol 169:2204 (2002). Moreover, in a Phase 2 trial drawn to the use of IL-10 for the treatment of psoriasis, hemoglobin, hematocrit, and erythrocyte counts were decreased during the course of therapy (Asadullah et al., Arch Dermatol 135:187-92 (1999). Thus, a nexus has been shown between elevated serum levels of IL-10 and toxicity associated with several hematological parameters.

The upper normal levels of these hematological parameters can be used as indicators of when toxicity might be observed. Clinicians generally consider normal values for red blood cell counts as the following: Males—4.32-5.72 trillion cells/L (4.32-5.72 million cells/mcL); Females—3.90-5.03 trillion cells/L (3.90-5.03 million cells/mcL). Clinicians generally consider normal values for hemoglobin as the following: Males—13.5-17.5 grams/dL (135-175 grams/L); Females—12.0-15.5 grams/dL (120-155 grams/L). Clinicians generally consider normal values for hematocrit as the following: Males—38.8-50.0 percent; Females −34.9-44.5 percent.

Other adverse effects have been observed during IL-10 therapy. Leukocytosis, white blood cell count above the normal range, has been observed in several clinical trials. Of the five principal types of leukocytosis (neutrophilia, lymphocytosis, monocytosis, eosinophilia, and basophilia), neutrophils were identified as being particularly responsible for the elevation in white blood cell counts. Further adverse effects associated with IL-10 include headache, nausea, fatigue, abdominal pain and back pain. [See, e.g., Asadullah et al., Arch Dermatol 135:187-92 (1999); Van Deventer et al., Gastroenterology 113:383-89 (1999)].

Subcutaneous Administration of IL-10:

Historically, subjects administered IL-10 agents for the treatment or prevention of various diseases, disorders, and conditions (e.g., cancer-related disorders, immune/inflammatory-related disorders, and metabolic-related disorders) have received the agents parenterally, including via intravenous and subcutaneous administration. As one would expect, compared to IV dosing, SC administration of IL-10 has been shown to result in extended exposure at the expense of peak exposure.

In the context of cholesterol lowering, the inventors have made the surprising discovery that SC administration of a particular dose of IL-10 is pharmacodynamically more active than a much larger dose administered IV, even when similar serum trough concentrations are compared. Although an understanding of the process underlying this finding is not needed in order to practice the present disclosure, one possible explanation is that SC administration results in the activation of a peripheral mechanism of action (e.g., activation of monocytic, myeloid or lymphocytic compartment) not seen with IV administration. In a particular embodiment, the present disclosure is based on the finding that there is an optimal dosing range that results in a therapeutically relevant reduction of serum cholesterol with minimum exposure. In another particular embodiment, the present disclosure is based on the finding that there is an optimal mean IL-10 serum trough concentration range that results in a therapeutically relevant reduction of serum cholesterol with minimum exposure.

IL-10 Serum Concentration:

The blood plasma levels of IL-10 in the methods described herein may be characterized in several manners, including: (1) a mean IL-10 serum trough concentration above some specified level or in a range of levels; (2) a mean IL-10 serum trough concentration above some specified level for some amount of time; (3) a steady state IL-10 serum concentration level above or below some specified level or in a range of levels; or (4) a Cₘₐₓ of the concentration profile above or below some specified level or in some range of levels. As set forth herein, mean serum trough IL-10 concentrations have been found to be of particular import for efficacy in certain indications.

In some embodiments of the present disclosure, blood plasma and/or serum level concentration profiles that may be produced include: a mean IL-10 plasma and/or serum trough concentration of greater than about 1.0 pg/mL, greater than about 10.0 pg/mL, greater than about 20.0 pg/mL, greater than about 30 pg/mL, greater than about 40 pg/mL, greater than about 50.0 pg/mL, greater than about 60.0 pg/mL, greater than about 70.0 pg/mL, greater than about 80.0 pg/mL, greater than about 90 pg/mL, greater than about 0.1 ng/mL, greater than about 0.2 ng/mL, greater than about 0.3 ng/mL, greater than about 0.4 ng/mL, greater than about 0.5 ng/mL, greater than about 0.6 ng/mL, greater than about 0.7 ng/mL, greater than about 0.8 ng/mL, greater than about 0.9 ng/mL, greater than about 1.0 ng/mL, greater than about 1.5 ng/mL, greater than about 2.0 ng/mL, greater than about 2.5 ng/mL, greater than about 3.0 ng/mL, greater than about 3.5 ng/mL, greater than about 4.0 ng/mL, greater than about 4.5 ng/mL, greater than about 5.0 ng/mL, greater than about 5.5 ng/mL, greater than about 6.0 ng/mL, greater than about 6.5 ng/mL, greater than about 7.0 ng/mL, greater than about 7.5 ng/mL, greater than about 8.0 ng/mL, greater than about 8.5 ng/mL, greater than about 9.0 ng/mL, greater than about 9.5 ng/mL, or greater than about 10.0 ng/mL.

In particular embodiments of the present disclosure, a mean IL-10 serum trough concentration is in the range of from 1.0 pg/mL to 10 ng/mL. In some embodiments, the mean IL-10 serum trough concentration is in the range of from 1.0 pg/mL to 100 pg/mL. In other embodiments, the mean IL-10 serum trough concentration is in the range of from 0.1 ng/mL to 1.0 ng/mL. In still other embodiments, the mean IL-10 serum trough concentration is in the range of from 1.0 ng/mL to 10 ng/mL. It is to be understood that the present disclosure contemplates ranges incorporating any concentrations encompassed by those set forth herein even if such ranges are not explicitly recited. By way of example, the mean serum IL-10 concentration in an embodiment may be in the range of from 0.1 ng/mL to 5 ng/mL. By way of further examples, particular embodiments of the present disclosure comprise a mean IL-10 serum trough concentration in a range of from about 0.1 ng/mL to about 10.5 ng/mL, from about 1.0 ng/mL to about 10.0 ng/mL, from about 1.0 ng/mL to about 9.0 ng/mL, from about 1.0 ng/mL to about 8.0 ng/mL, from about 1.0 ng/mL to about 7.0 ng/mL, from about 1.5 ng/mL to about 10.0 ng/mL, from about 1.5 ng/mL to about 9.0 ng/mL, from about 1.5 ng/mL to about 8.0 ng/mL, from about 1.5 ng/mL to about 7.0 ng/mL, from about 2.0 ng/mL to about 10.0 ng/mL, from about 2.0 ng/mL to about 9.0 ng/mL, from about 2.0 ng/mL to about 8.0 ng/mL, and from about 2.0 ng/mL to about 7.0 ng/mL.

In particular embodiments, a mean IL-10 serum trough concentration of 1-2 ng/mL is maintained over the duration of treatment. The present disclosure also contemplates embodiments wherein the mean IL-10 serum peak concentration is less than or equal to about 10.0 ng/mL over the duration of treatment. Further embodiments contemplate a mean IL-10 serum trough concentration greater than or equal to about 1.0 pg/mL. The optimal mean serum concentration is generally that at which the desired therapeutic effect is achieved without introducing undesired adverse effects. In most patient populations, maximum serum cholesterol reduction is frequently achieved at mean IL-10 serum trough concentrations of from about 1.0 ng/mL to about 10 ng/mL; at such concentrations, unacceptable adverse effects are generally not observed. However, lower mean Il-10 serum trough concentrations may also be advantageous in certain patient populations. For example, IL-10 serum trough concentrations of from about 0.1 ng/mL to about 1.0 ng/mL have been shown to decrease serum cholesterol levels by approximately 30%; such lower IL-10 serum trough concentrations might be a therapeutic goal in patients who exhibit adverse effects at higher concentrations. Moreover, low picogram per milliliter IL-10 serum levels may dramatically decrease arterial and other plaques.

Certain embodiments of the present disclosure provide a method for monitoring a subject receiving IL-10 therapy to predict, and thus potentially avoid, adverse effects, the method comprising: (1) measuring the subject's peak concentration of IL-10; (2) measuring the subject's trough concentration of IL-10; (3) calculating a peak-trough fluctuation; and, (4) using the calculated peak-trough fluctuation to predict potential adverse effects in the subject. In particular subject populations, a smaller peak-trough fluctuation indicates a lower probability that the subject will experience IL-10—related adverse effects. In addition, in some embodiments particular peak-trough fluctuations are determined for the treatment of particular diseases, disorders and conditions using particular dosing parameters, and those fluctuations are used as reference standards.

For the majority of drugs, plasma drug concentrations decline in a multi-exponential fashion. Immediately after intravenous administration, the drug rapidly distributes throughout an initial space (minimally defined as the plasma volume), and then a slower, equilibrative distribution to extravascular spaces (e.g., certain tissues) occurs. Intravenous IL-10 administration is associated with such a two-compartment kinetic model (see Rachmawati, H. et al. (2004) Pharm. Res. 21(11):2072-78).

Pharmacokinetic parameters following subcutaneous administration of recombinant human IL-10 have also been studied (Radwanski, E. et al. (1998) Pharm. Res. 15(12):1895-1901). As with many large macromolecules, it is believed that most of subcutaneously administered IL-10 is transported to the lymphatic circulation, which results in absorption-rate limited disposition and causes longer residence times than those observed with smaller macromolecules. Thus, the proposed pharmacokinetic model for subcutaneously administered IL-10 indicates that the agent initially forms a ‘depot’ at the injection site, similar to an initial loading dose phenomenon, from where a small amount is delivered rapidly and directly to the circulation by a zero-order process. The second phase of absorption entails a more gradual uptake of IL-10 in which the kinetics are first-order and governed mainly by the lymph flow.

In addition to the pharmacokinetic considerations outlined above for IL-10, the characteristics of pegylated IL-10 introduce additional considerations that are relevant to dosing parameters and regimens. Still further considerations may arise due to the nature of the disease, disorder or condition being treated, and whether the agent is being utilized as a therapeutic or as a prophylactic. All of these considerations provide opportunities for context-specific optimization of pegylated IL-10 treatment.

### Cholesterol and the Effect of PEG-IL-10 on Cholesterol Homeostasis and Indicators Thereof

Physiology:

Cholesterol plays an indispensable role in a vast array of physiological processes, including cell membrane structure, and biosynthesis of steroid hormones, bile acids and vitamin D. Cholesterol synthesis entails a complex 37-step process that begins with the reduction of 3-hydroxy-3-methylglutaryl CoA (HMG-CoA) to mevalonate by the enzyme HGM-CoA reductase. This is the regulated, rate-limiting and irreversible step in cholesterol synthesis and is the site of action for the statin drugs (HMG-CoA reductase competitive inhibitors).

The liver is the major regulator of cholesterol. Not only is it the site of formation of VLDL, the precursor of most LDL in the circulation, it is also the location where the vast majority of receptor-mediated clearance of LDL takes place. The liver initially clears all the cholesterol that is absorbed from the small intestine. Absorption of excess cholesterol may increase the amount of cholesterol stored in the liver, resulting in increased VLDL secretion (and thus LDL formation) and down-regulation of hepatic LDL-receptor activity. On average, about half of all cholesterol entering the intestine is absorbed. The fractional absorption rate varies greatly among individuals, which may explain, at least in part, why some patients respond poorly, or not at all, to statins and other classes of lipid-lowering drugs. See, e.g., Turley, S D, (2004) Clin. Cardiol. 6 Suppl 3:11116-21. The liver also recycles cholesterol by excreting it in a non-esterified form (via bile) into the digestive tract.

Lipid Panel:

Total cholesterol is defined as the sum of LDL, HDL, and VLDL. In general, total blood cholesterol levels <200 mg/dL are considered normal, levels between 200-239 mg/dL are considered borderline-high, and levels >240 mg/dL are considered high.

Since 1988, the National Cholesterol Education Program (NCEP) has issued guidelines identifying LDL as the primary target of cholesterol therapy. The current guidelines, set forth in Adult Treatment Panel-III (ATP-III), set a goal for LDL<100 mg/dL (2.6 mmol/L). Increased LDL is associated with atherosclerotic disease, which confers high risk for coronary heart disease (CHD)—related events, including clinical CHD, symptomatic carotid artery disease, peripheral arterial disease, and abdominal aortic aneurysm. Diseases, disorders and conditions associated with elevated cholesterol levels, and the treatment and/or prevention thereof, are described in detail hereafter.

There is considerable evidence indicating that low levels of high-density cholesterol (HDL-C, or simply HDL) are a contributory factor in the development of atherosclerosis and CHD. Low HDL is one of the most common lipid disorders in patients with premature coronary artery disease. Patients with hypertriglyceridemia usually have lower HDL cholesterol. Certain medications, including beta-blockers, progesterone and testosterone, also lower HDL levels.

In the average man, HDL cholesterol levels range from 40 to 50 mg/dL, whereas in the average woman, they range from 50 to 60 mg/dL. Studies have indicated that the median values of HDL associated with the lowest risk for atherosclerotic events are 62 mg/dL in men and 81 mg/dL in women. The ATP-III guidelines for lipid-lowing therapy established an HDL level below 40 mg/dL and an LDL level greater than or equal to 60 mg/dL as risk factors. A ratio of total cholesterol to HDL of less than 5:1 is considered to be desirable.

Triglycerides are predominantly carried in the blood stream by very low density lipoproteins (VLDL). There is considerable heterogeneity of triglyceride-rich particles. Triglyceride-rich particles derived from dietary fat—chylomicrons—are not themselves associated with CHD, but, when very high (>1,000 mg/dL), can cause pancreatitis, venous and arterial thrombi, acute heart attack and stroke. However, these chylomicron particles are gradually reduced in size by lipoprotein lipase to intermediate density lipoproteins (IDL) which are atherogenic. Similarly, VLDL from the liver is reduced in size by lipoprotein lipase, producing atherogenic IDL. VLDL is predictive of progression of coronary artery disease and CHD events, and thus hypertriglyceridemia has been increasingly recognized as a risk factor for CHD.

High triglyceride levels either result from genetic causes or are acquired. In terms of genetic causes, about 1/500 people have an inherited tendency towards high plasma triglycerides. Acquired high triglycerides are most commonly associated with excessive alcohol intake, exogenous estrogens or estrogen agonists, poorly controlled diabetes, beta-blockers, corticosteroids, and uremia. Triglycerides levels in excess of 1,000 mg/dL reflect an acquired cause for high triglycerides superimposed on a genetic cause. Less common causes of acquired high triglycerides include kidney failure, nephrotic syndrome, albuminuria, hypothyroidism, many liver diseases, hemochromatosis, hyperparathyroidism, and glycogen storage disease.

According to the American Heart Association, triglyceride levels of less than 150 mg/dL are normal; levels from 150 to 199 mg/dL are borderline high; levels from 200 to 499 mg/dL are high; and levels ≧500 mg/dL are very high. In general, triglyceride levels between 150 and 200 mg/dL are not pharmacologically treated.

Testing:

Several general methods and systems have been used in evaluating a subject's lipid profile. Any method or system, now in existence or subsequently developed, are contemplated herein and may be used in conjunction with the teachings of the present disclosure.

Fasting cholesterol tests, which generally utilize a colorimetric assay system, are the traditional means for measuring total serum cholesterol. Such tests require blood to be drawn after an approximately 12-hour fast to determine a lipoprotein profile. Usually, only the total cholesterol, HDL, and triglycerides are measured; for cost reasons, VLDL is usually estimated as one-fifth of the triglycerides and the LDL is estimated using the Friedewald formula. Although such tests are inexpensive and widely available (e.g., Sigma-Aldrich, St. Louis, Mo.; BioVision, Inc., Milpitas, Calif.), they require fasting and are not as sensitive as other tests because LDL is estimated rather than determined directly.

When assessing hypercholesterolemia, it is frequently useful to measure all lipoprotein subfractions (VLDL, IDL, LDL and HDL). Because a particular therapeutic goal is to decrease LDL (while maintaining or increasing HDL), cholesterol tests that directly measure LDL levels are more accurate, and they are especially useful for those patients who have elevated triglycerides. Though commercially available (e.g., Beckman Coulter, Inc.; Brea, Calif.), use of these direct measurement tests is sometimes limited due to their cost.

Effect of PEG-IL-10 on Cholesterol Homeostasis and Indicators Thereof:

In particular embodiments, an IL-10 agent disclosed herein (e.g., PEG-IL-10) has an anti-hyperlipidemia activity capable of reducing the levels of VLDL, IDL, LDL, or a combination thereof by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other embodiments, an IL-10 agent disclosed herein (e.g., PEG-IL-10) has anti-hyperlipidemia activity capable of reducing the levels of VLDL, IDL, LDL, or a combination thereof in a range from, e.g., about 10% to about 100%, about 20% to about 100%, about 30% to about 100%, about 40% to about 100%, about 50% to about 100%, about 60% to about 100%, about 70% to about 100%, or about 80% to about 100%; about 10% to about 90%, about 20% to about 90%, about 30% to about 90%, about 40% to about 90%, about 50% to about 90%, about 60% to about 90%, or about 70% to about 90%; about 10% to about 80%, about 20% to about 80%, about 30% to about 80%, about 40% to about 80%, about 50% to about 80%, or about 60% to about 80%; about 10% to about 70%, about 20% to about 70%, about 30% to about 70%, about 40% to about 70%, or about 50% to about 70%.

In another embodiment of the present disclosure, an IL-10 agent disclosed herein (e.g., PEG-IL-10) increases the level of HDL. In an aspect of this embodiment, the IL-10 agent increases the level of HDL by, e.g., at least 2%, at least 3%, at least 10%, at least 12%, at least 15%, at least 17%, at least 20%, at least 22%, at least 25%, at least 27%, at least 30%, at least 32%, at least 35%, at least 37%, at least 40%, at least 42%, at least 45% or at least 47%. In yet other embodiments of the present disclosure, the IL-10 agent increases the level of HDL in a range from, e.g., about 2% to about 100%; about 10% to about 50%, about 15% to about 50%, about 20% to about 50%, about 25% to about 50%, about 30% to about 50%, about 35% to about 50%, or about 40% to about 50%; about 2% to about 45%, about 10% to about 45%, about 15% to about 45%, about 20% to about 45%, about 25% to about 45%, about 30% to about 45%, or about 35% to about 45%; about 2% to about 40%, about 10% to about 40%, about 15% to about 40%, about 20% to about 40%, about 25% to about 40%, or about 30% to about 40%; about 2% to about 35%, about 10% to about 35%, about 15% to about 35%, about 20% to about 35%, or about 25% to about 35%.

It is to be understood that the aforementioned amounts, ranges, and the like are illustrative rather than limiting.

### Methods of Production of IL-10

A polypeptide of the present disclosure can be produced by any suitable method, including non-recombinant (e.g., chemical synthesis) and recombinant methods.

A. Chemical Synthesis

Where a polypeptide is chemically synthesized, the synthesis may proceed via liquid-phase or solid-phase. Solid-phase peptide synthesis (SPPS) allows the incorporation of unnatural amino acids and/or peptide/protein backbone modification. Various forms of SPPS, such as 9-fluorenylmethoxycarbonyl (Fmoc) and t-butyloxycarbonyl (Boc), are available for synthesizing polypeptides of the present disclosure. Details of the chemical syntheses are known in the art (e.g., Ganesan A. (2006) Mini Rev. Med. Chem. 6:3-10; and Camarero J. A. et al., (2005) Protein Pept Lett. 12:723-8).

Solid phase peptide synthesis may be performed as described hereafter. The alpha functions (Na) and any reactive side chains are protected with acid-labile or base-labile groups. The protective groups are stable under the conditions for linking amide bonds but can readily be cleaved without impairing the peptide chain that has formed. Suitable protective groups for the α-amino function include, but are not limited to, the following: Boc, benzyloxycarbonyl (Z), O-chlorbenzyloxycarbonyl, bi-phenylisopropyloxycarbonyl, tert-amyloxycarbonyl (Amoc), α, α-dimethyl-3,5-dimethoxy-benzyloxycarbonyl, o-nitrosulfenyl, 2-cyano-t-butoxy-carbonyl, Fmoc, 1-(4,4-dimethyl-2,6-dioxocylohex-1-ylidene)ethyl (Dde) and the like.

Suitable side chain protective groups include, but are not limited to: acetyl, allyl (All), allyloxycarbonyl (Alloc), benzyl (Bzl), benzyloxycarbonyl (Z), t-butyloxycarbonyl (Boc), benzyloxymethyl (Bom), o-bromobenzyloxycarbonyl, t-butyl (tBu), t-butyldimethylsilyl, 2-chlorobenzyl, 2-chlorobenzyloxycarbonyl, 2,6-dichlorobenzyl, cyclohexyl, cyclopentyl, 1-(4,4-dimethyl-2,6-dioxocyclohex-1-ylidene)ethyl (Dde), isopropyl, 4-methoxy-2,3-6-trimethylbenzylsulfonyl (Mtr), 2,3,5,7,8-pentamethylchroman-6-sulfonyl (Pmc), pivalyl, tetrahydropyran-2-yl, tosyl (Tos), 2,4,6-trimethoxybenzyl, trimethylsilyl and trityl (Trt).

In the solid phase synthesis, the C-terminal amino acid is coupled to a suitable support material. Suitable support materials are those which are inert towards the reagents and reaction conditions for the step-wise condensation and cleavage reactions of the synthesis process and which do not dissolve in the reaction media being used. Examples of commercially-available support materials include styrene/divinylbenzene copolymers which have been modified with reactive groups and/or polyethylene glycol; chloromethylated styrene/divinylbenzene copolymers; hydroxymethylated or aminomethylated styrene/divinylbenzene copolymers; and the like. When preparation of the peptidic acid is desired, polystyrene (1%)-divinylbenzene or TentaGel® derivatized with 4-benzyloxybenzyl-alcohol (Wang-anchor) or 2-chlorotrityl chloride can be used. In the case of the peptide amide, polystyrene (1%) divinylbenzene or TentaGel® derivatized with 5-(4′-aminomethyl)-3′,5′-dimethoxyphenoxy)valeric acid (PAL-anchor) or p-(2,4-dimethoxyphenyl-amino methyl)-phenoxy group (Rink amide anchor) can be used.

The linkage to the polymeric support can be achieved by reacting the C-terminal Fmoc-protected amino acid with the support material by the addition of an activation reagent in ethanol, acetonitrile, N,N-dimethylformamide (DMF), dichloromethane, tetrahydrofuran, N-methylpyrrolidone or similar solvents at room temperature or elevated temperatures (e.g., between 40° C. and 60° C.) and with reaction times of, e.g., 2 to 72 hours.

The coupling of the Nα-protected amino acid (e.g., the Fmoc amino acid) to the PAL, Wang or Rink anchor can, for example, be carried out with the aid of coupling reagents such as N,N′-dicyclohexylcarbodiimide (DCC), N,N′-diisopropylcarbodiimide (DIC) or other carbodiimides, 2-(1H-benzotriazol-1-yl)-1,1,3,3-tetramethyluronium tetrafluoroborate (TBTU) or other uronium salts, O-acyl-ureas, benzotriazol-1-yl-tris-pyrrolidino-phosphonium hexafluorophosphate (PyBOP) or other phosphonium salts, N-hydroxysuccinimides, other N-hydroxyimides or oximes in the presence or absence of 1-hydroxybenzotriazole or 1-hydroxy-7-azabenzotriazole, e.g., with the aid of TBTU with addition of HOBt, with or without the addition of a base such as, for example, diisopropylethylamine (DIEA), triethylamine or N-methylmorpholine, e.g., diisopropylethylamine with reaction times of 2 to 72 hours (e.g., 3 hours in a 1.5 to 3-fold excess of the amino acid and the coupling reagents, for example, in a 2-fold excess and at temperatures between about 10° C. and 50° C., for example, 25° C. in a solvent such as dimethylformamide, N-methylpyrrolidone or dichloromethane, e.g., dimethylformamide).

Instead of the coupling reagents, it is also possible to use the active esters (e.g., pentafluorophenyl, p-nitrophenyl or the like), the symmetric anhydride of the Nα-Fmoc-amino acid, its acid chloride or acid fluoride, under the conditions described above.

The Nα-protected amino acid (e.g., the Fmoc amino acid) can be coupled to the 2-chlorotrityl resin in dichloromethane with the addition of DIEA and having reaction times of 10 to 120 minutes, e.g., 20 minutes, but is not limited to the use of this solvent and this base.

The successive coupling of the protected amino acids can be carried out according to conventional methods in peptide synthesis, typically in an automated peptide synthesizer. After cleavage of the Nα-Fmoc protective group of the coupled amino acid on the solid phase by treatment with, e.g., piperidine (10% to 50%) in dimethylformamide for 5 to 20 minutes, e.g., 2×2 minutes with 50% piperidine in DMF and 1×15 minutes with 20% piperidine in DMF, the next protected amino acid in a 3 to 10-fold excess, e.g., in a 10-fold excess, is coupled to the previous amino acid in an inert, non-aqueous, polar solvent such as dichloromethane, DMF or mixtures of the two and at temperatures between about 10° C. and 50° C., e.g., at about 25° C. The previously mentioned reagents for coupling the first Nα-Fmoc amino acid to the PAL, Wang or Rink anchor are suitable as coupling reagents. Active esters of the protected amino acid, or chlorides or fluorides or symmetric anhydrides thereof, can also be used as an alternative.

At the end of the solid phase synthesis, the peptide is cleaved from the support material while simultaneously cleaving the side chain protecting groups. Cleavage can be carried out with trifluoroacetic acid or other strongly acidic media with addition of 5% to 20% V/V of scavengers such as dimethylsulfide, ethylmethylsulfide, thioanisole, thiocresol, m-cresol, anisole ethanedithiol, phenol or water, e.g., 15% v/v dimethylsulfide/ethanedithiol/m-cresol 1:1:1, within about 0.5 to 3 hours, e.g., 2 hours. Peptides with fully-protected side chains are obtained by cleaving the 2-chlorotrityl anchor with glacial acetic acid/trifluoroethanol/dichloromethane 2:2:6. The protected peptide can be purified by chromatography on silica gel. If the peptide is linked to the solid phase via the Wang anchor and if it is intended to obtain a peptide with a C-terminal alkylamidation, the cleavage can be carried out by aminolysis with an alkylamine or fluoroalkylamine. The aminolysis is carried out at temperatures between about −10° C. and 50° C. (e.g., about 25° C.), and reaction times between about 12 and 24 hours (e.g., about 18 hours). In addition the peptide can be cleaved from the support by re-esterification, e.g., with methanol.

The acidic solution that is obtained may be admixed with a 3- to 20-fold amount of cold ether or n-hexane, e.g., a 10-fold excess of diethyl ether, in order to precipitate the peptide and hence to separate the scavengers and cleaved protective groups that remain in the ether. A further purification can be carried out by re-precipitating the peptide several times from glacial acetic acid. The precipitate that is obtained can be taken up in water or tert-butanol or mixtures of the two solvents, e.g., a 1:1 mixture of tert-butanol/water, and freeze-dried.

The peptide obtained can be purified by various chromatographic methods, including ion exchange over a weakly basic resin in the acetate form; hydrophobic adsorption chromatography on non-derivatized polystyrene/divinylbenzene copolymers (e.g., Amberlite® XAD); adsorption chromatography on silica gel; ion exchange chromatography, e.g., on carboxymethyl cellulose; distribution chromatography, e.g., on Sephadex® G-25; countercurrent distribution chromatography; or high pressure liquid chromatography (HPLC) e.g., reversed-phase HPLC on octyl or octadecylsilylsilica (ODS) phases.

B. Recombinant Production

Methods describing the preparation of human and mouse IL-10 can be found in, for example, U.S. Pat. No. 5,231,012, which teaches methods for the production of proteins having IL-10 activity, including recombinant and other synthetic techniques. IL-10 can be of viral origin, and the cloning and expression of a viral IL-10 from Epstein Barr virus (BCRF1 protein) is disclosed in Moore et al., (1990) Science 248:1230. IL-10 can be obtained in a number of ways using standard techniques known in the art, such as those described herein. Recombinant human IL-10 is also commercially available, e.g., from PeproTech, Inc., Rocky Hill, N.J.

Where a polypeptide is produced using recombinant techniques, the polypeptide may be produced as an intracellular protein or as a secreted protein, using any suitable construct and any suitable host cell, which can be a prokaryotic or eukaryotic cell, such as a bacterial (e.g., E. coli) or a yeast host cell, respectively. Other examples of eukaryotic cells that may be used as host cells include insect cells, mammalian cells, and/or plant cells. Where mammalian host cells are used, they may include human cells (e.g., HeLa, 293, H9 and Jurkat cells); mouse cells (e.g., NIH3T3, L cells, and C127 cells); primate cells (e.g., Cos 1, Cos 7 and CV1); and hamster cells (e.g., Chinese hamster ovary (CHO) cells).

A variety of host-vector systems suitable for the expression of a polypeptide may be employed according to standard procedures known in the art. See, e.g., Sambrook et al., 1989 Current Protocols in Molecular Biology Cold Spring Harbor Press, New York; and Ausubel et al. 1995 Current Protocols in Molecular Biology, Eds. Wiley and Sons. Methods for introduction of genetic material into host cells include, for example, transformation, electroporation, conjugation, calcium phosphate methods and the like. The method for transfer can be selected so as to provide for stable expression of the introduced polypeptide-encoding nucleic acid. The polypeptide-encoding nucleic acid can be provided as an inheritable episomal element (e.g., a plasmid) or can be genomically integrated. A variety of appropriate vectors for use in production of a polypeptide of interest are commercially available.

Vectors can provide for extrachromosomal maintenance in a host cell or can provide for integration into the host cell genome. The expression vector provides transcriptional and translational regulatory sequences, and may provide for inducible or constitutive expression where the coding region is operably-linked under the transcriptional control of the transcriptional initiation region, and a transcriptional and translational termination region. In general, the transcriptional and translational regulatory sequences may include, but are not limited to, promoter sequences, ribosomal binding sites, transcriptional start and stop sequences, translational start and stop sequences, and enhancer or activator sequences. Promoters can be either constitutive (a strong constitutive promoter such as T7) or inducible.

Expression constructs generally have convenient restriction sites located near the promoter sequence to provide for the insertion of nucleic acid sequences encoding proteins of interest. A selectable marker operative in the expression host may be present to facilitate selection of cells containing the vector. Moreover, the expression construct may include additional elements. For example, the expression vector may have one or two replication systems, thus allowing it to be maintained in organisms, for example, in mammalian or insect cells for expression and in a prokaryotic host for cloning and amplification. In addition, the expression construct may contain a selectable marker gene to allow the selection of transformed host cells. Selectable genes are well known in the art and will vary with the host cell used.

Isolation and purification of a protein can be accomplished according to methods known in the art. For example, a protein can be isolated from a lysate of cells genetically modified to express the protein constitutively and/or upon induction, or from a synthetic reaction mixture by immunoaffinity purification, which generally involves contacting the sample with an anti-protein antibody, washing to remove non-specifically bound material, and eluting the specifically bound protein. The isolated protein can be further purified by dialysis and other methods normally employed in protein purification. In one embodiment, the protein may be isolated using metal chelate chromatography methods. Proteins may contain modifications to facilitate isolation.

The polypeptides may be prepared in substantially pure or isolated form (e.g., free from other polypeptides). The polypeptides can be present in a composition that is enriched for the polypeptide relative to other components that may be present (e.g., other polypeptides or other host cell components). For example, purified polypeptide may be provided such that the polypeptide is present in a composition that is substantially free of other expressed proteins, e.g., the other expressed proteins make up less than about 90%, less than about 60%, less than about 50%, less than about 40%, less than about 30%, less than about 20%, less than about 10%, less than about 5%, or less than about 1%.

An IL-10 polypeptide may be generated using recombinant techniques to manipulate different IL-10—related nucleic acids known in the art to provide constructs capable of encoding the IL-10 polypeptide. It will be appreciated that, when provided a particular amino acid sequence, the ordinary skilled artisan will recognize a variety of different nucleic acid molecules encoding such amino acid sequence in view of her background and experience in, for example, molecular biology.

### Amide Bond Substitutions

In some cases, IL-10 includes one or more linkages other than peptide bonds, e.g., at least two adjacent amino acids are joined via a linkage other than an amide bond. For example, in order to reduce or eliminate undesired proteolysis or other means of degradation, and/or to increase serum stability, and/or to restrict or increase conformational flexibility, one or more amide bonds within the backbone of IL-10 can be substituted.

In another example, one or more amide linkages (—CO—NH—) in IL-10 can be replaced with a linkage which is an isostere of an amide linkage, such as —CH₂NH—, —CH₂S—, —CH₂CH₂—, —CH═CH-(cis and trans), —COCH₂—, —CH(OH)CH₂— or —CH₂SO—. One or more amide linkages in IL-10 can also be replaced by, for example, a reduced isostere pseudopeptide bond. Such replacements and how to effect them are known to those of ordinary skill in the art. [See, e.g., Couder et al. (1993) Int. J. Peptide Protein Res. 41:181-184].

### Amino Acid Substitutions

One or more amino acid substitutions can be made in an IL-10 polypeptide. The following are non-limiting examples:

a) substitution of alkyl-substituted hydrophobic amino acids, including alanine, leucine, isoleucine, valine, norleucine, (S)-2-aminobutyric acid, (S)-cyclohexylalanine or other simple alpha-amino acids substituted by an aliphatic side chain from C₁-C₁₀ carbons including branched, cyclic and straight chain alkyl, alkenyl or alkynyl substitutions;

b) substitution of aromatic-substituted hydrophobic amino acids, including phenylalanine, tryptophan, tyrosine, sulfotyrosine, biphenylalanine, 1-naphthylalanine, 2-naphthylalanine, 2-benzothienylalanine, 3-benzothienylalanine, histidine, including amino, alkylamino, dialkylamino, aza, halogenated (fluoro, chloro, bromo, or iodo) or alkoxy (from C₁-C₄)-substituted forms of the above-listed aromatic amino acids, illustrative examples of which are: 2-, 3- or 4-aminophenylalanine, 2-, 3- or 4-chlorophenylalanine, 2-, 3- or 4-methylphenylalanine, 2-, 3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2, 3, or 4-biphenylalanine, 2′-, 3′-, or 4′-methyl-, 2-, 3- or 4-biphenylalanine, and 2- or 3-pyridylalanine;

c) substitution of amino acids containing basic side chains, including arginine, lysine, histidine, ornithine, 2,3-diaminopropionic acid, homoarginine, including alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma′-diethyl-homoarginine. Included also are compounds such as alpha-methyl-arginine, alpha-methyl-2,3-diaminopropionic acid, alpha-methyl-histidine, alpha-methyl-ornithine where the alkyl group occupies the pro-R position of the alpha-carbon. Also included are the amides formed from alkyl, aromatic, heteroaromatic (where the heteroaromatic group has one or more nitrogens, oxygens or sulfur atoms singly or in combination), carboxylic acids or any of the many well-known activated derivatives such as acid chlorides, active esters, active azolides and related derivatives, and lysine, ornithine, or 2,3-diaminopropionic acid;

d) substitution of acidic amino acids, including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids;

e) substitution of side chain amide residues, including asparagine, glutamine, and alkyl or aromatic substituted derivatives of asparagine or glutamine; and

f) substitution of hydroxyl-containing amino acids, including serine, threonine, homoserine, 2,3-diaminopropionic acid, and alkyl or aromatic substituted derivatives of serine or threonine.

In some cases, IL-10 comprises one or more naturally-occurring, non-genetically encoded L-amino acids, synthetic L-amino acids, or D-enantiomers of an amino acid. For example, IL-10 can comprise only D-amino acids. For example, an IL-10 polypeptide can comprise one or more of the following residues: hydroxyproline, β-alanine, o-aminobenzoic acid, m-aminobenzoic acid, p-aminobenzoic acid, m-aminomethylbenzoic acid, 2,3-diaminopropionic acid, α-aminoisobutyric acid, N-methylglycine (sarcosine), ornithine, citrulline, t-butylalanine, t-butylglycine, N-methylisoleucine, phenylglycine, cyclohexylalanine, norleucine, naphthylalanine, pyridylalanine 3-benzothienyl alanine, 4-chlorophenylalanine, 2-fluorophenylalanine, 3-fluorophenylalanine, 4-fluorophenylalanine, penicillamine, 1,2,3,4-tetrahydroisoquinoline-3-carboxylic acid, β-2-thienylalanine, methionine sulfoxide, homoarginine, N-acetyl lysine, 2,4-diamino butyric acid, rho-aminophenylalanine, N-methylvaline, homocysteine, homoserine, ε-amino hexanoic acid, ω-aminohexanoic acid, ω-aminoheptanoic acid, ω-aminooctanoic acid, ω-aminodecanoic acid, ω-aminotetradecanoic acid, cyclohexylalanine, α,γ-diaminobutyric acid, α,β-diaminopropionic acid, δ-amino valeric acid, and 2,3-diaminobutyric acid.

### Additional Modifications

One or more cysteine residues or cysteine analogs can be introduced into an IL-10 polypeptide to provide for linkage to another peptide via a disulfide bond or to provide for cyclization of the IL-10 polypeptide. Methods of introducing a cysteine or cysteine analog are known in the art; see, e.g., U.S. Pat. No. 8,067,532. Other means of cyclization include introduction of an oxime linker or a lanthionine linker; see, e.g., U.S. Pat. No. 8,044,175. Any combination of amino acids (or non-amino acid moieties) that can form a cyclizing bond can be used and/or introduced. A cyclizing bond can be generated with any combination of amino acids (or with an amino acid and —(CH2)ₙ—CO— or —(CH2)ₙ—C₆H₄—CO—) with functional groups which allow for the introduction of a bridge. Examples include disulfides, disulfide mimetics such as the —(CH2)ₙ— carba bridge, thioacetal, thioether bridges (cystathionine or lanthionine) and bridges containing esters and ethers. In these examples, n can be any integer, but is frequently less than ten.

Other modifications include, for example, an N-alkyl (or aryl) substitution (ψ[CONR]), or backbone crosslinking to construct lactams and other cyclic structures. Other derivatives include C-terminal hydroxymethyl derivatives, o-modified derivatives (e.g., C-terminal hydroxymethyl benzyl ether), N-terminally modified derivatives including substituted amides such as alkylamides and hydrazides.

In some cases, one or more L-amino acids in an IL-10 polypeptide is replaced with one or more D-amino acids.

In some cases, an IL-10 polypeptide is a retroinverso analog (see, e.g., Sela and Zisman (1997) FASEB J. 11:449). Retro-inverso peptide analogs are isomers of linear polypeptides in which the direction of the amino acid sequence is reversed (retro) and the chirality, D- or L-, of one or more amino acids therein is inverted (inverso), e.g., using D-amino acids rather than L-amino acids. [See, e.g., Jameson et al. (1994) Nature 368:744; and Brady et al. (1994) Nature 368:692].

An IL-10 polypeptide can include a “Protein Transduction Domain” (PTD), which refers to a polypeptide, polynucleotide, carbohydrate, or organic or inorganic molecule that facilitates traversing a lipid bilayer, micelle, cell membrane, organelle membrane, or vesicle membrane. A PTD attached to another molecule facilitates the molecule traversing a membrane, for example going from extracellular space to intracellular space, or cytosol to within an organelle. In some embodiments, a PTD is covalently linked to the amino terminus of an IL-10 polypeptide, while in other embodiments, a PTD is covalently linked to the carboxyl terminus of an IL-10 polypeptide. Exemplary protein transduction domains include, but are not limited to, a minimal undecapeptide protein transduction domain (corresponding to residues 47-57 of HIV-1 TAT comprising YGRKKRRQRRR; SEQ ID NO:3); a polyarginine sequence comprising a number of arginine residues sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); a Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:4); Transportan GWTLNSAGYLLGKINLKALAALAKKIL (SEQ ID NO:5); KALAWEAKLAKALAKALAKHLAKALAKALKCEA (SEQ ID NO:6); and RQIKIWFQNRRMKWKK (SEQ ID NO:7). Exemplary PTDs include, but are not limited to, an arginine homopolymer of from 3 arginine residues to 50 arginine residues; and the following exemplary PTD domain amino acid sequences:: YGRKKRRQRRR (SEQ ID NO:3); RKKRRQRRR (SEQ ID NO:8); RKKRRQRR (SEQ ID NO:9); YARAAARQARA (SEQ ID NO:10); THRLPRRRRRR (SEQ ID NO:11); and GGRRARRRRRR (SEQ ID NO:12).

The carboxyl group COR₃ of the amino acid at the C-terminal end of an IL-10 polypeptide can be present in a free form (R₃═OH) or in the form of a physiologically-tolerated alkaline or alkaline earth salt such as, e.g., a sodium, potassium or calcium salt. The carboxyl group can also be esterified with primary, secondary or tertiary alcohols such as, e.g., methanol, branched or unbranched C₁-C₆-alkyl alcohols, e.g., ethyl alcohol or tert-butanol. The carboxyl group can also be amidated with primary or secondary amines such as ammonia, branched or unbranched C₁-C₆-alkylamines or C₁-C₆ di-alkylamines, e.g., methylamine or dimethylamine.

The amino group of the amino acid NR₁R₂ at the N-terminus of an IL-10 polypeptide can be present in a free form (R₁═H and R₂═H) or in the form of a physiologically-tolerated salt such as, e.g., a chloride or acetate. The amino group can also be acetylated with acids such that R₁═H and R₂=acetyl, trifluoroacetyl, or adamantyl. The amino group can be present in a form protected by amino-protecting groups conventionally used in peptide chemistry, such as those provided above (e.g., Fmoc, Benzyloxy-carbonyl (Z), Boc, and Alloc). The amino group can be N-alkylated in which R₁ and/or R₂═C₁-C₆ alkyl or C₂-C₈ alkenyl or C₇-C₉ aralkyl. Alkyl residues can be straight-chained, branched or cyclic (e.g., ethyl, isopropyl and cyclohexyl, respectively).

Particular Modifications to Enhance and/or Mimic IL-10 Function

It is frequently beneficial, and sometimes imperative, to improve one of more physical properties of the treatment modalities disclosed herein (e.g., IL-10) and/or the manner in which they are administered. Improvements of physical properties include, for example, modulating immunogenicity; methods of increasing solubility, bioavailability, serum half-life, and/or therapeutic half-life; and/or modulating biological activity. Certain modifications may also be useful to, for example, raise of antibodies for use in detection assays (e.g., epitope tags) and to provide for ease of protein purification. Such improvements must generally be imparted without adversely impacting the bioactivity of the treatment modality and/or increasing its immunogenicity.

Pegylation of IL-10 is one particular modification contemplated by the present disclosure, while other modifications include, but are not limited to, glycosylation (N- and 0-linked); polysialylation; albumin fusion molecules comprising serum albumin (e.g., human serum albumin (HSA), cyno serum albumin, or bovine serum albumin (BSA)); albumin binding through, for example a conjugated fatty acid chain (acylation); and Fc-fusion proteins.

Pegylation:

The clinical effectiveness of protein therapeutics is often limited by short plasma half-life and susceptibility to protease degradation. Studies of various therapeutic proteins (e.g., filgrastim) have shown that such difficulties may be overcome by, for example, conjugating or linking the protein to any of a variety of nonproteinaceous polymers, e.g., polyethylene glycol (PEG), polypropylene glycol, or polyoxyalkylenes. This is frequently effected by a linking moiety covalently bound to both the protein and the nonproteinaceous polymer, e.g., a PEG. Such PEG-conjugated biomolecules have been shown to possess clinically useful properties, including better physical and thermal stability, protection against susceptibility to enzymatic degradation, increased solubility, longer in vivo circulating half-life and decreased clearance, reduced immunogenicity and antigenicity, and reduced toxicity.

In addition to the beneficial effects of pegylation on pharmacokinetic parameters, pegylation itself may enhance activity. For example, PEG-IL-10 has been shown to be more efficacious against certain cancers than unpegylated IL-10 (see, e.g., EP 206636A2).

PEGs suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure. A molecular weight of the PEG used in the present disclosure is not restricted to any particular range, and examples are set forth elsewhere herein; by way of example, certain embodiments have molecular weights between 5 kDa and 20 kDa, while other embodiments have molecular weights between 4 kDa and 10 kDa.

The present disclosure also contemplates compositions of conjugates wherein the PEGs have different n values, and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods know in the art. Exemplary reaction conditions are described throughout the specification. Cation exchange chromatography may be used to separate conjugates, and a fraction is then identified which contains the conjugate having, for example, the desired number of PEGs attached, purified free from unmodified protein sequences and from conjugates having other numbers of PEGs attached.

Pegylation most frequently occurs at the alpha amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry.

General pegylation strategies known in the art can be applied herein. PEG may be bound to a polypeptide of the present disclosure via a terminal reactive group (a “spacer” or “linker”) which mediates a bond between the free amino or carboxyl groups of one or more of the polypeptide sequences and polyethylene glycol. The PEG having the spacer which may be bound to the free amino group includes N-hydroxysuccinylimide polyethylene glycol which may be prepared by activating succinic acid ester of polyethylene glycol with N-hydroxysuccinylimide. Another activated polyethylene glycol which may be bound to a free amino group is 2,4-bis(O-methoxypolyethyleneglycol)-6-chloro-s-triazine, which may be prepared by reacting polyethylene glycol monomethyl ether with cyanuric chloride. The activated polyethylene glycol which is bound to the free carboxyl group includes polyoxyethylenediamine.

Conjugation of one or more of the polypeptide sequences of the present disclosure to PEG having a spacer may be carried out by various conventional methods. For example, the conjugation reaction can be carried out in solution at a pH of from 5 to 10, at temperature from 4° C. to room temperature, for 30 minutes to 20 hours, utilizing a molar ratio of reagent to protein of from 4:1 to 30:1. Reaction conditions may be selected to direct the reaction towards producing predominantly a desired degree of substitution. In general, low temperature, low pH (e.g., pH=5), and short reaction time tend to decrease the number of PEGs attached, whereas high temperature, neutral to high pH (e.g., pH≧7), and longer reaction time tend to increase the number of PEGs attached. Various means known in the art may be used to terminate the reaction. In some embodiments, the reaction is terminated by acidifying the reaction mixture and freezing at, e.g., −20° C. Pegylation of various molecules is discussed in, for example, U.S. Pat. Nos. 5,252,714; 5,643,575; 5,919,455; 5,932,462; and 5,985,263. PEG-IL-10 is described in, e.g., U.S. Pat. No. 7,052,686. Specific reaction conditions contemplated for use herein are set forth in the Experimental section.

The present disclosure also contemplates the use of PEG mimetics. Recombinant PEG mimetics have been developed that retain the attributes of PEG (e.g., enhanced serum half-life) while conferring several additional advantageous properties. By way of example, simple polypeptide chains (comprising, for example, Ala, Glu, Gly, Pro, Ser and Thr) capable of forming an extended conformation similar to PEG can be produced recombinantly already fused to the peptide or protein drug of interest (e.g., XTEN technology; Amunix; Mountain View, Calif.). This obviates the need for an additional conjugation step during the manufacturing process. Moreover, established molecular biology techniques enable control of the side chain composition of the polypeptide chains, allowing optimization of immunogenicity and manufacturing properties.

Glycosylation:

For purposes of the present disclosure, “glycosylation” is meant to broadly refer to the enzymatic process by which glycans are attached to proteins, lipids or other organic molecules. The use of the term “glycosylation” in conjunction with the present disclosure is generally intended to mean adding or deleting one or more carbohydrate moieties (either by removing the underlying glycosylation site or by deleting the glycosylation by chemical and/or enzymatic means), and/or adding one or more glycosylation sites that may or may not be present in the native sequence. In addition, the phrase includes qualitative changes in the glycosylation of the native proteins involving a change in the nature and proportions of the various carbohydrate moieties present.

Glycosylation can dramatically affect the physical properties (e.g., solubility) of polypeptides such as IL-10 and can also be important in protein stability, secretion, and subcellular localization. Glycosylated polypeptides may also exhibit enhanced stability or may improve one or more pharmacokinetic properties, such as half-life. In addition, solubility improvements can, for example, enable the generation of formulations more suitable for pharmaceutical administration than formulations comprising the non-glycosylated polypeptide.

Addition of glycosylation sites can be accomplished by altering the amino acid sequence. The alteration to the polypeptide may be made, for example, by the addition of, or substitution by, one or more serine or threonine residues (for O-linked glycosylation sites) or asparagine residues (for N-linked glycosylation sites). The structures of N-linked and O-linked oligosaccharides and the sugar residues found in each type may be different. One type of sugar that is commonly found on both is N-acetylneuraminic acid (hereafter referred to as sialic acid). Sialic acid is usually the terminal residue of both N-linked and O-linked oligosaccharides and, by virtue of its negative charge, may confer acidic properties to the glycoprotein. A particular embodiment of the present disclosure comprises the generation and use of N-glycosylation variants.

The polypeptide sequences of the present disclosure may optionally be altered through changes at the nucleic acid level, particularly by mutating the nucleic acid encoding the polypeptide at preselected bases such that codons are generated that will translate into the desired amino acids.

Polysialylation:

The present disclosure also contemplates the use of polysialylation, the conjugation of polypeptides to the naturally occurring, biodegradable α-(2→8) linked polysialic acid (“PSA”) in order to improve the polypeptides' stability and in vivo pharmacokinetics.

Albumin Fusion:

Additional suitable components and molecules for conjugation include albumins such as human serum albumin (HSA), cyno serum albumin, and bovine serum albumin (BSA).

According to the present disclosure, albumin may be conjugated to a drug molecule (e.g., a polypeptide described herein) at the carboxyl terminus, the amino terminus, both the carboxyl and amino termini, and internally (see, e.g., U.S. Pat. No. 5,876,969 and U.S. Pat. No. 7,056,701).

In the HSA—drug molecule conjugates contemplated by the present disclosure, various forms of albumin may be used, such as albumin secretion pre-sequences and variants thereof, fragments and variants thereof, and HSA variants. Such forms generally possess one or more desired albumin activities. In additional embodiments, the present disclosure involves fusion proteins comprising a polypeptide drug molecule fused directly or indirectly to albumin, an albumin fragment, and albumin variant, etc., wherein the fusion protein has a higher plasma stability than the unfused drug molecule and/or the fusion protein retains the therapeutic activity of the unfused drug molecule. In some embodiments, the indirect fusion is effected by a linker, such as a peptide linker or modified version thereof.

As alluded to above, fusion of albumin to one or more polypeptides of the present disclosure can, for example, be achieved by genetic manipulation, such that the nucleic acid coding for HSA, or a fragment thereof, is joined to the nucleic acid coding for the one or more polypeptide sequences.

Alternative Albumin Binding Strategies:

Several albumin—binding strategies have been developed as alternatives to direct fusion and may be used with the IL-10 agents described herein. By way of example, the present disclosure contemplates albumin binding through a conjugated fatty acid chain (acylation) and fusion proteins which comprise an albumin binding domain (ABD) polypeptide sequence and the sequence of one or more of the polypeptides described herein.

Conjugation with Other Molecules:

Additional suitable components and molecules for conjugation include, for example, thyroglobulin; tetanus toxoid; Diphtheria toxoid; polyamino acids such as poly(D-lysine:D-glutamic acid); VP6 polypeptides of rotaviruses; influenza virus hemaglutinin, influenza virus nucleoprotein; Keyhole Limpet Hemocyanin (KLH); and hepatitis B virus core protein and surface antigen; or any combination of the foregoing.

Thus, the present disclosure contemplates conjugation of one or more additional components or molecules at the N- and/or C-terminus of a polypeptide sequence, such as another polypeptide (e.g., a polypeptide having an amino acid sequence heterologous to the subject polypeptide), or a carrier molecule. Thus, an exemplary polypeptide sequence can be provided as a conjugate with another component or molecule.

An IL-10 polypeptide may also be conjugated to large, slowly metabolized macromolecules such as proteins; polysaccharides, such as sepharose, agarose, cellulose, or cellulose beads; polymeric amino acids such as polyglutamic acid, or polylysine; amino acid copolymers; inactivated virus particles; inactivated bacterial toxins such as toxoid from diphtheria, tetanus, cholera, or leukotoxin molecules; inactivated bacteria; and dendritic cells. Such conjugated forms, if desired, can be used to produce antibodies against a polypeptide of the present disclosure.

Additional candidate components and molecules for conjugation include those suitable for isolation or purification. Particular non-limiting examples include binding molecules, such as biotin (biotin-avidin specific binding pair), an antibody, a receptor, a ligand, a lectin, or molecules that comprise a solid support, including, for example, plastic or polystyrene beads or plates, magnetic beads, test strips, and membranes.

Fc-fusion Molecules:

In certain embodiments, the amino- or carboxyl-terminus of a polypeptide sequence of the present disclosure can be fused with an immunoglobulin Fc region (e.g., human Fc) to form a fusion conjugate (or fusion molecule). Fc fusion conjugates have been shown to increase the systemic half-life of biopharmaceuticals, and thus the biopharmaceutical product may require less frequent administration.

Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates.

Other Modifications:

The present disclosure contemplates the use of other modifications, currently known or developed in the future, of IL-10 to improve one or more properties. Examples include hesylation, various aspects of which are described in, for example, U.S. Patent Appln. Nos. 2007/0134197 and 2006/0258607, and fusion molecules comprising SUMO as a fusion tag (LifeSensors, Inc.; Malvern, Pa.).

Linkers:

Linkers and their use have been described above. Any of the foregoing components and molecules used to modify the polypeptide sequences of the present disclosure may optionally be conjugated via a linker. Suitable linkers include “flexible linkers” which are generally of sufficient length to permit some movement between the modified polypeptide sequences and the linked components and molecules. The linker molecules are generally about 6-50 atoms long. The linker molecules may also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids.

Exemplary flexible linkers include glycine polymers (G)ₙ, glycine-serine polymers (for example, (GS)ₙ, GSGGSₙ (SEQ ID NO:13), and GGGSₙ (SEQ ID NO:14), where n is an integer of at least one), glycine-alanine polymers, alanine-serine polymers, and other flexible linkers. Glycine and glycine-serine polymers are relatively unstructured, and therefore may serve as neutral tethers between components. Exemplary flexible linkers include, but are not limited to, GGSG (SEQ ID NO:15), GGSGG (SEQ ID NO:16), GSGSG (SEQ ID NO:17), GSGGG (SEQ ID NO:18), GGGSG (SEQ ID NO:19), and GSSSG (SEQ ID NO:20).

Further examples of flexible linkers include glycine polymers (G)ₙ, glycine-alanine polymers, alanine-serine polymers, glycine-serine polymers (for example, (GₘSₒ)ₙ, (GSGGS)ₙ (SEQ ID NO:21), (GₘSₒGₘ)ₙ, (GₘSₒGₘSₒGₘ)ₙ (SEQ ID NO:22), (GSGGSₘ)ₙ (SEQ ID NO:23), (GSGSₘG)ₙ (SEQ ID NO:24) and (GGGSₘ)ₙ (SEQ ID NO:25), and combinations thereof, where m, n, and o are each independently selected from an integer of at least 1 to 20, e.g., 1-18, 2-16, 3-14, 4-12, 5-10, 1, 2, 3, 4, 5, 6, 7, 8, 9, or 10), and other flexible linkers. Glycine and glycine-serine polymers are relatively unstructured, and therefore may serve as a neutral tether between components. Examples of flexible linkers include, but are not limited to GGSG (SEQ ID NO:15), GGSGG (SEQ ID NO:16), GSGSG (SEQ ID NO:17), GSGGG (SEQ ID NO:18), GGGSG (SEQ ID NO:19), and GSSSG (SEQ ID NO:20).

Additional flexible linkers include glycine polymers (G)ₙ or glycine-serine polymers (e.g., (GS)ₙ, (GSGGS)ₙ (SEQ ID NO:26), (GGGS)ₙ (SEQ ID NO:27) and (GGGGS)ₙ (SEQ ID NO:28), where n=1 to 50, for example, 1, 2, 3, 4, 5, 6, 7, 8, 9, 10, 10-20, 20-30, 30-50. Exemplary flexible linkers include, but are not limited to GGGS (SEQ ID NO:29), GGGGS (SEQ ID NO:30), GGSG (SEQ ID NO:15), GGSGG (SEQ ID NO:16), GSGSG (SEQ ID NO:17), GSGGG (SEQ ID NO:18), GGGSG (SEQ ID NO:19), and GSSSG (SEQ ID NO:20). A multimer (e.g., 1, 2, 3, 4, 5, 6, 7, 8, 9, 10, 10-20, 20-30, or 30-50) of these linker sequences may be linked together to provide flexible linkers that may be used to conjugate a heterologous amino acid sequence to the polypeptides disclosed herein. As described herein, the heterologous amino acid sequence may be a signal sequence and/or a fusion partner, such as, albumin, Fc sequence, and the like.

### Antibodies

The present disclosure also contemplates antibodies in certain embodiments. For example, an antibody therapeutic agent may be used in combination with an IL-10 agent. As alluded to elsewhere herein, the term “antibody” encompasses intact monoclonal antibodies, polyclonal antibodies, multispecific antibodies (e.g., bispecific antibodies) formed from at least two intact antibodies, and antibody binding fragments including Fab and F(ab)′₂, provided that they exhibit the desired biological activity.

The present disclosure contemplates antibodies generated in any manner (currently used or that may be used in the future), including fully human and humanized antibodies. The skilled artisan is aware of various methods of generating such antibodies. By way of example, fully human monoclonal antibodies can be prepared by the generation of hybridoma cell lines or by the use of sequences encoding particular antibodies for transformation of a suitable mammalian host cell, such as a CHO cell.

As used herein, the term “epitope” refers to binding sites for antibodies on protein antigens. Epitopic determinants usually comprise chemically active surface groupings of molecules such as amino acids or sugar side chains, as well as specific three-dimensional structural and charge characteristics. An antibody is said to bind an antigen when the dissociation constant is ≦1 μM, ≦100 nM, or ≦10 nM. An increased equilibrium constant (“K_{D}”) means that there is less affinity between the epitope and the antibody, whereas a decreased equilibrium constant means that there is more affinity between the epitope and the antibody. An antibody with a K_{D} of “no more than” a certain amount means that the antibody will bind to the epitope with the given K_{D} or more strongly. Whereas K_{D} describes the binding characteristics of an epitope and an antibody, “potency” describes the effectiveness of the antibody itself for a function of the antibody. There is not necessarily a correlation between an equilibrium constant and potency; thus, for example, a relatively low K_{D} does not automatically mean a high potency.

The term “selectively binds” in reference to an antibody does not mean that the antibody only binds to a single substance, but rather that the K_{D} of the antibody to a first substance is less than the K_{D} of the antibody to a second substance. An antibody that exclusively binds to an epitope only binds to that single epitope.

### Therapeutic and Prophylactic Uses

The present disclosure contemplates the subcutaneous administration of the IL-10 polypeptides described herein (e.g., PEG-IL-10) in the treatment and/or prevention of diseases, disorders or conditions, and/or the symptoms thereof, relating to, or resulting from, for example, hypercholesterolemia, aberrant lipid profile, and other disorders associated, directly or indirectly, with cholesterol homeostasis. While particular uses are described in detail hereafter, it is to be understood that the present disclosure is not so limited. In addition, although specific categories of exemplary diseases, disorders and conditions associated with, or resulting from, hypercholesterolemia and aberrant lipid profile are discussed hereafter, it is to be understood that there is often overlap between one or more categories (e.g., certain cardiovascular diseases may have an inflammatory component).

Cardiovascular Diseases.

In particular embodiments, the present disclosure contemplates the use of the IL-10 polypeptides (e.g., PEG-IL-10) described herein to treat and/or prevent cardiovascular diseases, disorders and conditions, as well as disorders associated therewith, resulting from hypercholesterolemia and aberrant lipid profile.

As used herein, the terms “cardiovascular disease”, “heart disease” and the like refer to any disease that affects the cardiovascular system, primarily cardiac disease, vascular diseases of the brain and kidney, and peripheral arterial diseases. Cardiovascular disease is a constellation of diseases, some of which are discussed further hereafter, that includes coronary heart disease (e.g., ischemic heart disease or coronary artery disease), atherosclerosis, cardiomyopathy, hypertension, hypertensive heart disease, cor pulmonale, cardiac dysrhythmias, endocarditis, cerebrovascular disease, and peripheral arterial disease. Cardiovascular disease is the leading cause of deaths worldwide, and while it usually affects older adults, the antecedents of cardiovascular disease, notably atherosclerosis, begin in early life.

In certain embodiments, the present disclosure contemplates the treatment and/or prevention of a peripheral vascular disease (PVD), also known as peripheral arterial disease (PAD) or peripheral arterial occlusive disease (PAOD). PVDs refer broadly to conditions characterized by an obstruction of large arteries, not within the coronary or cerebral vasculature, which results in either acute or chronic ischemia. PVDs also include a subset of diseases classified as microvascular diseases resulting from episodic narrowing of the arteries (e.g., Raynaud's phenomenon) or widening of the arteries (e.g., a vascular spasm). Symptoms of PVDs include, without limitation, pain, weakness, numbness, cramping in muscles due to decreased blood flow, sores, wounds, ulcers that heal slowly or not at all, and limb coolness or discoloration. About 20% of patients with mild PAD may be asymptomatic.

In particular embodiments, the IL-10 agents (e.g., PEG-IL-10) of the present disclosure are used in the treatment and/or prevention of a cardiovascular disease that comprises a cardiomyopathy, a condition characterized by the deterioration of myocardium function. Signs and symptoms may mimic those of almost any form of heart disease and include chest pain and EKG abnormalities. A mild cardiomyopathy is frequently asymptomatic, whereas a severe case is associated with heart failure, arrhythmias, systemic embolization or sudden cardiac death.

Several schemes may be used to classify a cardiomyopathy. One scheme classifies a cardiomyopathy functionally, as involving dilation, hypertrophy, or restriction. Another scheme classifies a cardiomyopathy as either extrinsic or intrinsic. An extrinsic cardiomyopathy refers to a cardiomyopathy where the primary pathology is outside the myocardium itself. For example, an extrinsic cardiomyopathy may be caused by a metabolic/storage disorder, an endocrine disorder, a neuromuscular disorder, a nutritional disorder, an inflammatory disorder, a toxicity (including drug and alcohol), an ischemia, and/or an infection (e.g., Hepatitis C). Non-limiting examples of extrinsic cardiomyopathies include acromegaly, alcoholic cardiomyopathy, amyloidosis, Chagas disease, diabetic cardiomyopathy, hemochromatosis, hypertensive cardiomyopathy, hyperthyroidism, inflammatory cardiomyopathy, ischemic cardiomyopathy, muscular dystrophy, valvular cardiomyopathy, a cardiomyopathy secondary to a systemic metabolic disease, a cardiomyopathy secondary to a systemic nutritional disease, a coronary artery disease, and a congenital heart disease. In contrast, an intrinsic cardiomyopathy refers to a cardiomyopathy characterized by weakness in the heart muscle that is of unknown origin. Non-limiting examples of intrinsic cardiomyopathies include dilated cardiomyopathy, hypertrophic cardiomyopathy, arrhythmogenic right ventricular cardiomyopathy, restrictive cardiomyopathy, isolated ventricular non-compaction, mitochondrial myopathy, Takotsubo cardiomyopathy, and Loeffler endocarditis.

Embodiments contemplated by the present disclosure include those wherein the disclosed IL-10 agents (e.g., PEG-IL-10) are used in the treatment and/or prevention of: i) an ischemic heart disease or myocardial ischemia, a condition characterized by reduced blood supply of the heart muscle, usually due to a narrowing or blockage of a coronary artery. Symptoms of ischemic heart disease include chest pain on exertion, in cold weather, or emotional situations; acute chest pain; acute coronary syndrome; unstable angina; myocardial infarction; heart failure, difficulty in breathing; or swelling of the extremities; ii) a congestive heart failure, conditions characterized by a heart abnormality that impairs the ability of the heart to fill with or pump a sufficient amount of blood throughout the body; and iii) a hypertensive heart disease, conditions characterized by high blood pressure that include, without limitation, left ventricular hypertrophy, coronary heart disease, congestive heart failure, hypertensive cardiomyopathy, and cardiac arrhythmias.

Particular embodiments of the present disclosure are directed to the use of the IL-10 polypeptides described herein to treat and/or prevent atherosclerosis, a chronic condition in which an arterial wall thickens to form plaques as a result of the accumulation of fatty materials such as cholesterol and triglycerides. Atherosclerosis frequently involves a chronic inflammatory response in the walls of arteries, caused largely by the accumulation of macrophages and promoted by LDLs without adequate removal of fats and cholesterol from the macrophages by functional HDLs. Chronically expanding atherosclerotic lesions can cause complete closure of the lumen, which may only manifest when the lumen stenosis is so severe that blood supply to downstream tissue(s) is insufficient, resulting in ischemia.

Particularly contemplated by the present disclosure are embodiments wherein the cardiovascular disease comprises a hyperlipidemia (or hyperlipoproteinemia), conditions characterized by abnormally elevated levels of lipids and/or lipoproteins in the blood. Hyperlipidemias may be classified as familial (or primary) when caused by specific genetic abnormalities, acquired (or secondary) when resulting from another underlying disorder, or idiopathic, when of unknown cause. Hyperlipidemias may also be classified based on which types of lipids and/or lipoproteins are elevated. Non-limiting examples of hyperlipidemias include dyslipidemia, hypercholesterolemia, hyperglyceridemia, hypertriglyceridemia, hyperlipoproteinemia, hyperchylomicronemia, and combined hyperlipidemia. Hyperlipoproteinemias include, for example, hyperlipoproteinemia type Ia, hyperlipoproteinemia type Ib, hyperlipoproteinemia type Ic, hyperlipoproteinemia type IIa, hyperlipoproteinemia type IIb, hyperlipoproteinemia type III, hyperlipoproteinemia type IV, and hyperlipoproteinemia type V.

Attempts to treat cardiovascular disease by controlling levels of lipids and/or lipoproteins in the blood have met with limited success. For example, although administration of statins reduces cardiovascular risk in some individuals, these therapeutic compounds do not reduce triglyceride levels. In individuals at cardiovascular risk who exhibit deleteriously high levels of triglycerides, a member of the fibrate class of therapeutic agents may be administered. However, although they lower triglyceride and LDL levels, fibrates do not affect HDL levels. Moreover, combination treatments involving statins and fibrates, while sometimes effective, often cause a significant increase in the risk of myopathy and rhabdomyolysis, and therefore can only be used under very close medical supervision. In view of limitations as exemplified above, there is clearly a need for improved agents for the use and treatment of cardiovascular diseases, including those associated with high lipid and/or lipoprotein levels.

Thrombosis and Thrombotic Conditions.

In other embodiments, the present disclosure contemplates the use of the IL-10 polypeptides (e.g., PEG-IL-10) described herein to treat and/or prevent thrombosis and thrombotic diseases, disorders and conditions, as well as disorders associated therewith, resulting from hypercholesterolemia and aberrant lipid profile. Thrombosis, the formation of a thrombus inside a blood vessel resulting in obstruction of the flow of blood through the circulatory system, may be caused by abnormalities in one or more of the following (Virchow's triad): hypercoagulability or increased blood clotting, endothelial cell injury, or disturbed blood flow (stasis, turbulence).

Thrombosis is generally categorized as venous or arterial, each of which can be presented by several subtypes. Venous thrombosis includes deep vein thrombosis (DVT), portal vein thrombosis, renal vein thrombosis, jugular vein thrombosis, Budd-Chiari syndrome, Paget-Schroetter disease, and cerebral venous sinus thrombosis. Arterial thrombosis includes stroke and myocardial infarction.

Inflammatory Disorders.

When cholesterol and/or LDL become embedded in the walls of blood vessels, an immune response can be triggered, which, in turn, results in chronic inflammation. In response to this inflammation, blood monocytes adhere to the endothelium, transmigrate into the subendothelial space, and differentiate toward macrophages. Macrophages, in turn, engulf the cholesterol deposits and modified LDL by phagocytosis via scavenger receptors, which are distinct from LDL receptors. However, the adaptive mechanisms mediated by macrophages are not sufficient to process the uncontrolled cholesterol and/or LDL deposition seen under pathologic conditions. As a result, the lipid-laden macrophages transform into “foam cells”, often accompanied by release of inflammation-inducing molecules. Both cholesterol/LDL deposition and the attendant foam cell-mediated pro-inflammatory reactions in the walls of the blood vessels lead to the development of atherosclerotic lesions. Thus, one consequence of modulating the levels of a lipid or lipoprotein is the reduction or elimination of a chronic inflammation.

The present disclosure includes embodiments wherein the IL-10 agents described herein (e.g., PEG-IL-10) are used in the treatment and/or prevention of a vasculitis. Vasculitis is a varied group of disorders featuring inflammation of a vessel wall, including lymphatic vessels and blood vessels like veins (phlebitis), arteries (arteritis) and capillaries, due to leukocyte migration and resultant damage. The inflammation may affect arteries and/or veins, regardless of size. It may be focal or widespread, with areas of inflammation scattered throughout a particular organ or tissue, or even affecting more than one organ system in the body. Vasculitis includes, without limitation, Buerger's disease (thromboangiitis obliterans), cerebral vasculitis (central nervous system vasculitis), Churg-Strauss arteritis, cryoglobulinemia, essential cryoglobulinemic vasculitis, giant cell (temporal) arteritis, Henoch-Schonlein purpura, hypersensitivity vasculitis (allergic vasculitis), Kawasaki disease, microscopic polyarteritis/polyangiitis, polyarteritis nodosa, polymyalgia rheumatica (PMR), rheumatoid vasculitis, Takayasu arteritis, thrombophlebitis, Wegener's granulomatosis; and vasculitis secondary to connective tissue disorders like systemic lupus erythematosus, rheumatoid arthritis, relapsing polychondritis, Behcet's disease, or other connective tissue disorders; and vasculitis secondary to viral infection.

Other embodiments are directed to an inflammatory heart disease, which refers to a condition characterized by inflammation of the heart muscle and/or the surrounding tissue. Examples include, but are not limited to, endocarditis, inflammatory cardiomegaly, and myocarditis.

### Pharmaceutical Compositions

The IL-10 polypeptides of the present disclosure may be in the form of compositions suitable for administration to a subject. In general, such compositions are “pharmaceutical compositions” comprising IL-10 and one or more pharmaceutically acceptable or physiologically acceptable diluents, carriers or excipients. Unless indicated otherwise, the terms “pharmaceutical” and “physiological” are used interchangeably. In certain embodiments, the IL-10 polypeptides are present in a therapeutically acceptable amount (e.g. an amount identified using the methods disclosed herein). The pharmaceutical compositions may be used in the methods of the present disclosure; thus, for example, the pharmaceutical compositions can be administered ex vivo or in vivo to a subject in order to practice the therapeutic and prophylactic methods and uses described herein.

The pharmaceutical compositions of the present disclosure can be formulated to be compatible with the intended method or route of administration; exemplary routes of administration (e.g., subcutaneous) are set forth herein. Furthermore, the pharmaceutical compositions may be used in combination with other therapeutically active agents or compounds as described herein in order to treat or prevent the diseases, disorders and conditions as contemplated by the present disclosure.

The pharmaceutical compositions typically comprise a therapeutically effective amount of an IL-10 polypeptide contemplated by the present disclosure and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water-soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), and N-tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampoule, syringe, or autoinjector (similar to, e.g., an EpiPen®)), whereas a multi-use container (e.g., a multi-use vial) is provided in other embodiments. Any drug delivery apparatus may be used to deliver the IL-10 agents, including implants (e.g., implantable pumps) and catheter systems, slow injection pumps and devices, all of which are well known to the skilled artisan. Depot injections, which are generally administered subcutaneously or intramuscularly, may also be utilized to release the polypeptides disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or

oleagenous suspension. This suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor EL™ (BASF, Parsippany, N.J.) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose any bland fixed oil may be employed, including synthetic mono- or diglycerides. Moreover, fatty acids such as oleic acid, find use in the preparation of injectables. Prolonged absorption of particular injectable formulations can be achieved by including an agent that delays absorption (e.g., aluminum monostearate or gelatin).

The pharmaceutical compositions containing the active component may be in a form suitable for oral use, for example, as tablets, capsules, troches, lozenges, aqueous or oily suspensions, dispersible powders or granules, emulsions, hard or soft capsules, syrups, solutions, microbeads or elixirs. In particular embodiments, an active component of an agent co-administered with an IL-10 agent described herein is in the form of a pharmaceutical composition suitable for oral use. Pharmaceutical compositions intended for oral use may be prepared according to any method known to the art for the manufacture of pharmaceutical compositions, and such compositions may contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for the manufacture of tablets. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration may be uncoated or coated by known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents to control delivery of an administered composition include biodegradable or biocompatible particles or a polymeric substance such as polyesters, polyamine acids, hydrogel, polyvinyl pyrrolidone, polyanhydrides, polyglycolic acid, ethylene-vinylacetate, methylcellulose, carboxymethylcellulose, protamine sulfate, or lactide/glycolide copolymers, polylactide/glycolide copolymers, or ethylenevinylacetate copolymers. For example, the oral agent can be entrapped in microcapsules prepared by coacervation techniques or by interfacial polymerization, by the use of hydroxymethylcellulose or gelatin-microcapsules or poly (methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents, such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified herein.

The pharmaceutical compositions of the present disclosure may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

Formulations can also include carriers to protect the composition against rapid degradation or elimination from the body, such as a controlled release formulation, including implants, liposomes, hydrogels, prodrugs and microencapsulated delivery systems. For example, a time delay material such as glyceryl monostearate or glyceryl stearate alone, or in combination with a wax, may be employed.

The present disclosure contemplates pharmaceutical compositions in a form suitable for rectal administration. By way of example, an agent for use in combination with an IL-10 polypeptide of the present disclosure (or an IL-10 polypeptide itself) may be in the form of a suppository. The suppositories can be prepared by mixing the drug with a suitable non-irritating excipient which is solid at ordinary temperatures but liquid at rectal temperature and will therefore melt in the rectum to release the drug. Such materials include, but are not limited to, cocoa butter and polyethylene glycols.

The IL-10 polypeptides contemplated by the present disclosure may be in the form of any other suitable pharmaceutical composition (e.g., sprays for nasal or inhalation use) currently known or developed in the future.

The concentration of a polypeptide or fragment thereof in a formulation can vary widely (e.g., from less than about 0.1%, usually at or at least about 2% to as much as 20% to 50% or more by weight) and can be selected based on fluid volumes, viscosities, and subject-based factors in accordance with, for example, the particular mode of administration selected.

### Routes of Administration

The present disclosure contemplates the administration of IL-10 agents, active therapeutic agents used in combination with IL-10 agents, and compositions of the foregoing, in any appropriate manner. Suitable routes of administration include parenteral (e.g., intramuscular, intravenous, subcutaneous (e.g., injection or implant), intraperitoneal, intracisternal, intraarticular, intraperitoneal, intracerebral (intraparenchymal) and intracerebroventricular), oral, nasal, vaginal, sublingual, intraocular, rectal, topical (e.g., transdermal), sublingual and inhalation. Depot injections, which are generally administered subcutaneously or intramuscularly, may also be utilized to release the IL-10 polypeptides disclosed herein over a defined period of time.

Particular embodiments of the present disclosure contemplate parenteral administration, and in further particular embodiments the parenteral administration is subcutaneous.

### Combination Therapy

The present disclosure contemplates the use of IL-10 (e.g., PEG-IL-10) in combination with one or more active therapeutic agents or other prophylactic or therapeutic modalities (e.g., radiation). In such combination therapy, the various active agents frequently have different mechanisms of action than IL-10. Such combination therapy may be especially advantageous by allowing a dose reduction of one or more of the agents, thereby reducing or eliminating the adverse effects associated with one or more of the agents; furthermore, such combination therapy may have a synergistic therapeutic or prophylactic effect on the underlying disease, disorder, or condition.

In particular embodiments, the present disclosure provides methods for treating and/or preventing diseases, disorders or conditions associated with (either directly or indirectly) cholesterol homeostasis, including associated cardiovascular, thrombotic and inflammatory disorders, with the IL-10 polypeptides described herein (e.g., PEG-IL-10) and at least one additional therapeutic or diagnostic agent. It is to be understood that combination therapy is not limited to agents that treat and/or prevent the aforementioned diseases, disorders and conditions (e.g., cholesterol-related disorders); for example, agents contemplated for use in combination with the IL-10 polypeptides may have efficacy in treating or preventing other metabolic disorders, such as diabetes or obesity. Use of the IL-10 polypeptides (e.g., PEG-IL-10) in combination with modified diets and/or exercise regimens is also contemplated herein.

As used herein, “combination” is meant to include therapies that can be administered separately, for example, formulated separately for separate administration (e.g., as may be provided in a kit), and therapies that can be administered together in a single formulation (i.e., a “co-formulation”).

In certain embodiments, the IL-10 polypeptides are administered or applied sequentially, e.g., where one therapeutic agent is administered prior to one or more other therapeutic agents. In other embodiments, the IL-10 polypeptides are administered simultaneously, e.g., where two or more therapeutic agents are administered at or about the same time; the two or more agents may be present in two or more separate formulations or combined into a single formulation (i.e., a co-formulation). Regardless of whether the two or more therapeutic agents are administered sequentially or simultaneously, they are considered to be administered in combination for purposes of the present disclosure.

The IL-10 polypeptides of the present disclosure may be used in combination with at least one therapeutic agent in any manner appropriate under the circumstances. In one embodiment, treatment with the at least one therapeutic agent and at least one IL-10 polypeptide of the present disclosure is maintained over a period of time. In another embodiment, treatment with the at least one therapeutic agent is reduced or discontinued (e.g., when the subject is stable), while treatment with the IL-10 polypeptide of the present disclosure is maintained at a constant dosing regimen. In a further embodiment, treatment with the at least one therapeutic agent is reduced or discontinued (e.g., when the subject is stable), while treatment with the IL-10 polypeptide of the present disclosure is reduced (e.g., lower dose, less frequent dosing or shorter treatment regimen). In yet another embodiment, treatment with the at least one therapeutic agent is reduced or discontinued (e.g., when the subject is stable), and treatment with the IL-10 polypeptide of the present disclosure is increased (e.g., higher dose, more frequent dosing or longer treatment regimen). In yet another embodiment, treatment with the at least one therapeutic agent is maintained and treatment with the IL-10 polypeptide of the present disclosure is discontinued or reduced (e.g., lower dose, less frequent dosing or shorter treatment regimen). In yet another embodiment, treatment with the at least one therapeutic agent and treatment with the IL-10 polypeptide of the present disclosure are discontinued or reduced (e.g., lower dose, less frequent dosing or shorter treatment regimen).

While particular therapeutic agents suitable for use in combination with the IL-10 polypeptides (e.g., PEG-IL-10) disclosed herein are set forth hereafter, it is to be understood that the present disclosure is not so limited. Hereafter, certain therapeutic agents are set forth in specific categories of exemplary diseases, disorders and conditions; however, it is to be understood that there is often overlap between one or more categories (e.g., certain therapeutic agents may have both cardiovascular and anti-inflammatory effects).

Cholesterol Homeostasis Agents.

Particular embodiments of the present disclosure involve combinations of IL-10 polypeptides with therapeutic agents associated with cholesterol homeostasis. Many of these therapeutic agents target different pathways involving the absorption, synthesis, transport, storage, catabolism, and excretion of cholesterol, and are thus particularly useful candidates for combination therapy.

Examples of therapeutic agents useful in combination therapy for the treatment of hypercholesterolemia (and thus frequently atherosclerosis, for example) include statins (e.g., CRESTOR™, LESCOL™, LIPITOR™, MEVACOR™, PRAVACOL™, and ZOCOR™), which inhibit the enzymatic synthesis of cholesterol; bile acid resins (e.g., COLESTID™, LO-CHOLEST™, PREVALITE™, QUESTRAN™, and WELCHOL™), which sequester cholesterol and prevent its absorption; ezetimibe (ZETIA™), which blocks cholesterol absorption; fibric acid (e.g., TRICOR™), which reduces triglycerides and may modestly increase HDL; niacin (e.g., NIACOR™), which modestly lowers LDL cholesterol and triglycerides; and/or a combination of the aforementioned (e.g., VYTORIN™ (ezetimibe with simvastatin). Alternative cholesterol treatments that may be candidates for use in combination with the IL-10 polypeptides described herein include various supplements and herbs (e.g., garlic, policosanol, and guggul). Several classes of the aforementioned therapeutic agents are discussed further hereafter.

Particular embodiments of the present disclosure comprise an IL-10 agent in combination with a fibrate. Fibrates, a class of amphipathic carboxylic acids, may be used as anti-hyperlipidemic agents to decrease levels of, e.g., triglycerides and LDL, and to increase levels of HDL. Examples of suitable fibrates include, without limitation, Bezafibrate, Ciprofibrate, Clofibrate, Gemfibrozil, and Fenofibrate.

Further particular embodiments of the present disclosure comprise an IL-10 agent in combination with a HMG-CoA Reductase Inhibitor (a statin). HMG-CoA Reductase Inhibitors may lower LDL and/or cholesterol levels by inhibiting the enzyme HMG-CoA Reductase, which plays a central role in the production of cholesterol in the liver. To compensate for the decreased cholesterol availability, synthesis of hepatic LDL receptors is increased, resulting in increased clearance of LDL particles from the blood. Examples of suitable statins include, without limitation, Atorvastatin, Fluvastatin, Lovastatin, Pitavastatin, Pravastatin, Rosuvastatin, and Simvastatin. Combinations of IL-10 polypeptides with a statin are particularly contemplated herein.

Still further particular embodiments of the present disclosure comprise an IL-10 agent in combination with a niacin. Niacins may lower LDL levels by selectively inhibiting hepatic diacyglycerol acyltransferase-2; reducing triglyceride synthesis; and reducing VLDL secretion through a receptor HM74 and HM74A or GPR109A. A non-limiting use of a niacin is as an anti-hyperlipidemic agent to inhibit the breakdown of fats in adipose tissue. By blocking the breakdown of fats, a niacin causes a decrease in free fatty acids in the blood and, as a consequence, decreases the secretion of VLDL and cholesterol by the liver. By lowering VLDL levels, a niacin may also increase the level of HDL in blood. Examples of suitable niacins include, without limitation, acipimox, niacin, nicotinamide, and vitamin B3.

Other particular embodiments of the present disclosure comprise an IL-10 agent in combination with a bile acid sequestrant. Bile acid sequestrants (resins) bind certain components of bile in the gastrointestinal tract, thereby disrupting the enterohepatic circulation of bile acids and preventing their reabsorption from the gut. Bile acid sequestrants are particularly effective for lowering LDL and cholesterol, and may also raise HDL levels. Examples of suitable bile acid sequestrants include, without limitation, Cholestyramine, Colesevelam, and Colestipol.

Additional particular embodiments of the present disclosure comprise an IL-10 agent in combination with a cholesterol absorption inhibitor. Cholesterol absorption inhibitors decrease absorption of cholesterol from the intestine; this leads to up-regulation of LDL-receptors on the surface of cells and increased LDL cholesterol uptake into these cells, thus decreasing levels of LDL in the blood plasma. Examples of suitable cholesterol absorption inhibitors include, without limitation, ezetimibe, a phytosterol, a sterol and a stanol. Combinations of IL-10 polypeptides with ezetimibe are particularly contemplated herein. Ezetimibe selectively blocks cholesterol absorption and lowers plasma LDL levels by an average of 18%. When ezetimibe is co-administered with lower doses of statins, there is an additive reduction in LDL levels, which equals the reduction achieved with maximal doses of statins alone. Reduction in the statin dose results in fewer statin-related adverse effects.

Still further particular embodiments of the present disclosure comprise an IL-10 agent in combination with a fat absorption inhibitor. Fat absorption inhibitors decrease the absorption of fat from the intestine, thereby reducing caloric intake. In one aspect, a fat absorption inhibitor inhibits pancreatic lipase, an enzyme that breaks down triglycerides in the intestine. Examples of suitable fat absorption inhibitors include, without limitation, Orlistat.

In still other particular embodiments, the present disclosure contemplates use of the PEG-IL-10 agents described herein in combination with modulators of PCSK9 (Proprotein convertase subtilisin/kexin type 9), a serine protease expressed predominantly in the liver, intestine and kidney. As part of the cholesterol homeostasis process, LDL cholesterol is removed from the blood when it binds to LDL receptors (LDLR) on the surface of liver cells and is taken up by such cells. PCSK9 functions by binding to LDLR and inducing receptor degradation, thereby preventing LDLR recycling to the cell surface to remove more LDL cholesterol, ultimately resulting in decreased metabolism thereof. Preventing PCSK9 binding to LDLR allows the receptor to return to the cell surface and remove more cholesterol. PEG-IL-10 down-regulates message expression of PCSK9 in knock-out mice lacking the LDL receptor. Thus, PEG-IL-10 dramatically lowers cholesterol in a non-PCSK9—dependent manner, indicating that combination of PEG-IL-10 with a PCSK9 inhibitor would have additive effects.

Inhibitors of PCSK9 function have been shown to cause much more cholesterol lowering than traditional commercially available agents, with an acceptable adverse effect profile. The present disclosure contemplates the use of PEG-IL-10 with any modulator having a direct or indirect inhibitory effect on PCSK9. Several monoclonal antibodies that bind to PCSK9 and interfere with its interaction with the LDLR are being developed (e.g., Amgen (AMG145), Merck (1D05-IgG2) and Aventis/Regeneron (SAR236553/REGN727)). In addition, peptides that mimic the EGFA domain of the LDLR that binds to PCSK9 are being developed, and gene silencing through the administration of a PCSK9 antisense oligonucleotide (ISIS Pharmaceuticals) has been shown to increase expression of the LDLR and decrease circulating total cholesterol levels in mice. Other modulators of PCSK9 function contemplated for combination therapy with the IL-10 agents (e.g., PEG-IL-10) described herein are those which act by means of RNA interference (RNAi) (Alnylam Pharmaceuticals) and as a locked nucleic acid (LNA) (Santaris Pharma), also referred to as inaccessible RNA.

The present disclosure encompasses pharmaceutically acceptable salts, acids or derivatives of any of the above.

Immune and Inflammatory Conditions.

In some embodiments, the present disclosure provides methods comprising subcutaneous administration of an IL-10 agent for the treatment of a cholesterol-related disease, disorder or condition in combination with one or more therapeutic agents for treating and/or preventing immune- and/or inflammatory-related diseases, disorders and conditions, as well as disorders associated therewith. By way of example, an IL-10 polypeptide may be administered with a therapeutic agent having efficacy in a cardiovascular disorder having an inflammatory component.

Examples of therapeutic agents useful in combination therapy include, but are not limited to, non-steroidal anti-inflammatory drugs (NSAIDs). NSAIDs, a large group of therapeutic compounds with analgesic, anti-inflammatory, and anti-pyretic properties, reduce inflammation by blocking cyclooxygenase. Examples of such agents include ibuprofen, and other propionic acid derivatives (alminoprofen, benoxaprofen, bucloxic acid, carprofen, fenbufen, fenoprofen, fluprofen, flurbiprofen, indoprofen, ketoprofen, miroprofen, naproxen, oxaprozin, pirprofen, pranoprofen, suprofen, tiaprofenic acid, and tioxaprofen); acetic acid derivatives (indomethacin, acemetacin, alclofenac, clidanac, diclofenac, fenclofenac, fenclozic acid, fentiazac, fuirofenac, ibufenac, isoxepac, oxpinac, sulindac, tiopinac, tolmetin, zidometacin, and zomepirac); fenamic acid derivatives (flufenamic acid, meclofenamic acid, mefenamic acid, niflumic acid and tolfenamic acid); biphenylcarboxylic acid derivatives (diflunisal and flufenisal); oxicams (isoxicam, piroxicam, sudoxicam and tenoxican); salicylates (acetyl salicylic acid, sulfasalazine); and the pyrazolones (apazone, bezpiperylon, feprazone, mofebutazone, oxyphenbutazone, phenylbutazone).

Other combinations include selective cyclooxygenase-2 (COX-2) inhibitors, selective cyclooxygenase 1 (COX 1) inhibitors, and non-selective cyclooxygenase (COX) inhibitors. Particular embodiments of the present disclosure contemplate the IL-10 polypeptides described herein (e.g., PEG-IL-10) in combination with a suitable selective COX-2 inhibitor(s), such as Celecoxib, Etoricoxib, Firocoxib, Lumiracoxib, Meloxicam, Parecoxib, Rofecoxib, and Valdecoxib.

Other therapeutic agents for combination therapy include steroids such as prednisolone, prednisone, methylprednisolone, betamethasone, dexamethasone, or hydrocortisone. Such a combination may be especially advantageous, since one or more side-effects of the steroid can be reduced or even eliminated by decreasing the steroid dose required when treating patients in combination with the present IL-10 polypeptides.

Additional examples of therapeutic agents contemplated for use in combination with the IL-10 agents (e.g., PEG-IL-10) disclosed herein are agents useful for treating, for example, rheumatoid arthritis and include cytokine suppressive anti-inflammatory drug(s) (CSAIDs); antibodies to or antagonists of other human cytokines or growth factors, for example, TNF, LT, IL-1β., IL-2, IL-6, IL-7, IL-8, IL-15, IL-16, IL-18, EMAP-II, GM-CSF, FGF, or PDGF.

Particular combinations of active agents may interfere at different points in the autoimmune and subsequent inflammatory cascade, and include TNF antagonists like chimeric, humanized or human TNF antibodies, REMICADE™, anti-TNF antibody fragments (e.g., CDP870), and soluble p55 or p75 TNF receptors, derivatives thereof, p75TNFRIgG (ENBREL™) or p55TNFR1gG (LENERCEPT™), soluble IL-13 receptor (sIL-13), and also TNFα converting enzyme (TACE) inhibitors; similarly IL-1 inhibitors (e.g., Interleukin-1-converting enzyme inhibitors) may be effective. Other combinations include Interleukin 11, anti-P7s and p-selectin glycoprotein ligand (PSGL). Other examples of therapeutic agents useful in combination with the IL-10 polypeptides described herein include interferon-β1a (AVONEX™); interferon-β1b (BETASERON™); copaxone; hyperbaric oxygen; intravenous immunoglobulin; clabribine; and antibodies to or antagonists of other human cytokines or growth factors (e.g., antibodies to CD40 ligand and CD80).

The present disclosure encompasses pharmaceutically acceptable salts, acids or derivatives of any of the above.

Anti-diabetic and Anti-obesity Agents.

Some subjects requiring pharmacological treatment for a cholesterol-related disorder(s) are also taking anti-diabetic and/or anti-obesity agents. The present disclosure contemplates combination therapy with numerous anti-diabetic agents (and classes thereof), including 1) insulin, insulin mimetics and agents that entail stimulation of insulin secretion, including sulfonylureas (e.g., chlorpropamide, tolazamide, acetohexamide, tolbutamide, glyburide, glimepiride, glipizide) and meglitinides (e.g., repaglinide (PRANDIN™) and nateglinide (STARLIX™)); 2) biguanides (e.g., metformin (GLUCOPHAGE™)) and other agents that act by promoting glucose utilization, reducing hepatic glucose production and/or diminishing intestinal glucose output; 3) alpha-glucosidase inhibitors (e.g., acarbose and miglitol) and other agents that slow down carbohydrate digestion and consequently absorption from the gut and reduce postprandial hyperglycemia; 4) thiazolidinediones (e.g., rosiglitazone (AVANDIA™), troglitazone (REZULIN™), pioglitazone (ACTOS™), glipizide, balaglitazone, rivoglitazone, netoglitazone, troglitazone, englitazone, ciglitazone, adaglitazone, and darglitazone) that enhance insulin action (e.g., by insulin sensitization), thus promoting glucose utilization in peripheral tissues; 5) glucagon-like-peptides, including DPP-IV inhibitors (e.g., vildagliptin (GALVUS™) and sitagliptin (JANUVIA™)) and Glucagon-Like Peptide-1 (GLP-1) and GLP-1 agonists and analogs (e.g., exenatide (BYETTA™)); 6) and DPP-IV-resistant analogues (incretin mimetics), PPAR gamma agonists, dual-acting PPAR agonists, pan-acting PPAR agonists, PTP1B inhibitors, SGLT inhibitors, insulin secretagogues, glycogen synthase kinase-3 inhibitors, immune modulators, beta-3 adrenergic receptor agonists, 11beta-HSD1 inhibitors, and amylin analogues. In still other embodiments, the IL-10 agents described herein are used in combination with one or more suitable nuclear receptor binding agents (e.g., a Retinoic Acid Receptor (RAR) binding agent, a Retinoid X Receptor (RXR) binding agent, a Liver X Receptor (LXR) binding agent and a Vitamin D binding agent).

Furthermore, the present disclosure contemplates combination therapy with agents and methods for promoting weight loss, such as agents that stimulate metabolism or decrease appetite, and modified diets and/or exercise regimens to promote weight loss.

The present disclosure encompasses pharmaceutically acceptable salts, acids or derivatives of any of the above.

### Dosing

The IL-10 agents (e.g., PEG-IL-10) of the present disclosure may be administered to a subject in an amount that is dependent upon, for example, the goal of the administration (e.g., the degree of resolution desired); the age, weight, sex, and health and physical condition of the subject the formulation being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan. Knowledge of the pharmacokinetic and pharmacodynamic parameters associated with ADME is useful in determining such amounts.

The present disclosure contemplates administration of IL-10 to achieve and/or maintain certain serum concentrations, such as trough concentrations or peak concentrations. Methodologies specific to IL-10 are described elsewhere herein and in this section below.

In general, dosing parameters dictate that the dosage amount be less than an amount that could be irreversibly toxic to the subject (i.e., the maximum tolerated dose, “MTD”) and not less than an amount required to produce a measurable effect on the subject. The therapeutic index (also known as therapeutic ratio) is a comparison of the amount of a therapeutic agent that causes the desired therapeutic effect to the amount that causes death (in animal studies) or toxicity (in human studies). Quantitatively, it is the ratio given by the lethal or toxic dose divided by the therapeutic dose. The therapeutic index can be determined in animal studies by dividing the lethal dose of therapeutic agent for 50% of the population (LD₅₀) by the minimum effective dose (i.e., a therapeutic response or desired effect) for 50% of the population (ED₅₀), whereas in human studies, the dose that produces a toxicity in 50% of the population (TD₅₀) is used instead of the LD₅₀ to calculate the therapeutic index. A higher therapeutic index is preferable to a lower one, as a subject would have to take a much higher dose of such a drug to reach the lethal/toxic threshold than the dose taken to elicit the therapeutic effect.

Although the ED₅₀ is commonly used as a measure of reasonable expectance of an agent's effect, it is not necessarily the dose that a clinician might deem appropriate taking into consideration all relevant factors. Thus, in some situations the effective amount is more than the calculated ED₅₀, in other situations the effective amount is less than the calculated ED₅₀, and in still other situations the effective amount is the same as the calculated ED₅₀.

In addition, an effective dose of the IL-10 polypeptides of the present disclosure may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

The amount of PEG-IL-10 necessary to treat a disease, disorder or condition described herein (e.g., a cholesterol-related disorder) is based on the IL-10 activity of the conjugated protein, which can be determined by IL-10 activity assays known in the art. By way of example, in the tumor context, suitable IL-10 activity includes, for example, CD8+ T-cell infiltrate into tumor sites, expression of inflammatory cytokines, such as IFN-γ, IL-4, IL-6, IL-10, and RANK-L, from these infiltrating cells, and increased levels of TNF-α or IFN-γ in biological samples.

The present disclosure contemplates the use of any amount of an IL-10 agent (e.g., PEG-IL-10) that can be administered subcutaneously for the treatment of the cholesterol-related diseases, disorders or conditions described elsewhere herein (e.g., from about 0.1 to about 80 μg/kg of body weight/day; from about 0.5 to about 10 μg/kg of body weight/day, or about 1 to 4 μg protein/kg of body weight/day), or for the prevention of any such diseases, disorders or conditions. Exemplary amounts are set forth hereafter. Though generally discussed in the context of treatment of cholesterol-related disorders, such amounts may also be applicable to non-cholesterol-related disorders.

The therapeutically effective amount of PEG-IL-10 can range from about 0.01 to about 100 μg protein/kg of body weight/day; from about 0.1 to about 80 μg/kg of body weight/day; from about 0.1 to about 60 μg/kg of body weight/day; or from about 0.1 to about 40 μg/kg of body weight/day. The therapeutically effective amount of PEG-IL-10 can also range from about 0.5 to about 100 μg protein/kg of body weight/day; from about 0.5 to about 80 μg/kg of body weight/day; from about 0.5 to about 60 μg/kg of body weight/day; or from about 0.5 to about 40 μg/kg of body weight/day. In some embodiments, the therapeutically effective amount of PEG-IL-10 can range from about 1.0 to about 100 μg protein/kg of body weight/day; from about 1.0 to about 80 μg/kg of body weight/day; from about 1.0 to about 60 μg/kg of body weight/day; or from about 1.0 to about 40 μg/kg of body weight/day. In still further embodiments, The therapeutically effective amount of PEG-IL-10 can range from about 5.0 to about 100 μg protein/kg of body weight/day; from about 5.0 to about 80 μg/kg of body weight/day; from about 5.0 to about 60 μg/kg of body weight/day; or from about 5.0 to about 40 μg/kg of body weight/day. The therapeutically effective amount of PEG-IL-10 can range from about 10.0 to about 100 μg protein/kg of body weight/day; from about 10.0 to about 80 μg/kg of body weight/day; from about 10.0 to about 60 μg/kg of body weight/day; or from about 10.0 to 40 μg/kg of body weight/day.

The therapeutically effective amount of PEG-IL-10 can range from about 0.01 to about 50 μg protein/kg of body weight/day; from about 0.1 to about 25 μg/kg of body weight/day; from about 0.1 to about 10 μg/kg of body weight/day; or from about 0.1 to about 5.0 μg/kg of body weight/day. The therapeutically effective amount of PEG-IL-10 can also range from about 0.5 to about 50 μg protein/kg of body weight/day; from about 0.5 to about 25 μg/kg of body weight/day; from about 0.5 to about 10 μg/kg of body weight/day; or from about 0.5 to about 5.0 μg/kg of body weight/day. In some embodiments, the therapeutically effective amount of PEG-IL-10 can range from about 1.0 to about 50 μg protein/kg of body weight/day; from about 1.0 to about 25 μg/kg of body weight/day; from about 1.0 to about 10 μg/kg of body weight/day; or from about 1.0 to about 5.0 μg/kg of body weight/day. In still further embodiments, the therapeutically effective amount of PEG-IL-10 can range from about 5.0 to about 50 μg protein/kg of body weight/day; from about 5.0 to about 25 μg/kg of body weight/day; or from about 5.0 to about 10 μg/kg of body weight/day. The therapeutically effective amount of PEG-IL-10 can range from about 10.0 to about 50 μg protein/kg of body weight/day; from about 10.0 to about 40 μg/kg of body weight/day; from about 10.0 to about 30 μg/kg of body weight/day; or from about 10.0 to 20 μg/kg of body weight/day.

In some embodiments, PEG-IL-10 is administered by continuous infusion to delivery about 50 to 800 μg protein/kg of body weight/day (e.g., about 1 to 16 μg protein/kg of body weight/day of PEG-IL-10). The infusion rate may be varied based on evaluation of, for example, adverse effects and blood cell counts.

For administration of an oral agent (e.g., a therapeutic agent for use in combination with an IL-10 agent described herein), the pharmaceutical compositions can be provided in and dosage form described herein, including tablets, capsules and the like containing from 1.0 to 1000 milligrams of the active therapeutic agent, particularly 1.0, 3.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active therapeutic agent.

In certain embodiments, the dosage of the disclosed IL-10 agents (e.g., PEG-IL-10) is contained in a “unit dosage form”. The phrase “unit dosage form” refers to physically discrete units, each unit containing a predetermined amount of an IL-10 agent of the present disclosure, either alone or in combination with one or more additional therapeutic agents, sufficient to produce the desired effect. It will be appreciated that the parameters of a unit dosage form will depend on the particular agent and the effect to be achieved.

### Kits

The present disclosure also contemplates kits comprising the IL-10, agents (e.g., PEG-IL-10) described herein, and pharmaceutical compositions thereof. The kits are generally in the form of a physical structure housing various components, as described below, and may be utilized, for example, in practicing the methods described above (e.g., administration of a IL-10 polypeptide to a subject in need of restoring cholesterol homeostasis).

A kit can include one or more of the IL-10 polypeptides disclosed herein (provided in, e.g., a sterile container), which may be in the form of a pharmaceutical composition suitable for administration to a subject. The IL-10 polypeptides can be provided in a form that is ready for use or in a form requiring, for example, reconstitution or dilution prior to administration. When the IL-10 polypeptides are in a form that needs to be reconstituted by a user, the kit may also include buffers, pharmaceutically acceptable excipients, and the like, packaged with or separately from the IL-10 polypeptides. When combination therapy is contemplated, the kit may contain the several therapeutic agents separately or they may already be combined in the kit. Each component of the kit may be enclosed within an individual container, and all of the various containers may be within a single package. A kit of the present disclosure may be designed for conditions necessary to properly maintain the components housed therein (e.g., refrigeration or freezing).

A kit may contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampoule, tube or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via the internet, are provided.

### Experimental

The following examples are put forth so as to provide those of ordinary skill in the art with a complete disclosure and description of how to make and use the present invention, and are not intended to limit the scope of what the inventors regard as their invention nor are they intended to represent that the experiments below were performed and are all of the experiments that may be performed. It is to be understood that exemplary descriptions written in the present tense were not necessarily performed, but rather that the descriptions can be performed to generate the data and the like described therein. Efforts have been made to ensure accuracy with respect to numbers used (e.g., amounts, temperature, etc.), but some experimental errors and deviations should be accounted for.

Unless indicated otherwise, parts are parts by weight, molecular weight is weight average molecular weight, temperature is in degrees Celsius (° C.), and pressure is at or near atmospheric. Standard abbreviations are used, including the following: bp=base pair(s); kb=kilobase(s); s or sec=second(s); min=minute(s); h or hr=hour(s); aa=amino acid(s); kb=kilobase(s); nt=nucleotide(s); pg=picogram; ng=nanogram; μg=microgram; mg=milligram; g=gram; kg=kilogram; pl or pL=picoliter(s); dl or dL=deciliter; μl or μL=microliter; ml or mL=milliliter; 1 or L=liter; μM=micromolar; mM=millimolar; M=molar; kDa=kilodalton; i.m.=intramuscular(ly); i.p.=intraperitoneal(ly); SC or SQ=subcutaneous(ly); QD=daily; BID=twice daily; QW=weekly; TIW=three times a week; QM=monthly; HPLC=high performance liquid chromatography; BW=body weight; U=unit; ns=not statistically significant; PBS=phosphate-buffered saline; PCR=polymerase chain reaction; NHS═N-Hydroxysuccinimide; HSA=human serum albumin; BSA=bovine serum albumin; DMEM=Dulbeco's Modification of Eagle's Medium; GC=genome copy; EDTA=ethylenediaminetetraacetic acid.

### Materials and Methods

The following general materials and methods were used, where indicated, or may be used in the Examples below:

Standard methods in molecular biology are described in the scientific literature (see, e.g., Sambrook and Russell (2001) Molecular Cloning, 3^{rd} ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)).

The scientific literature describes methods for protein purification, including immunoprecipitation, chromatography, electrophoresis, centrifugation, and crystallization, as well as chemical analysis, chemical modification, post-translational modification, production of fusion proteins, and glycosylation of proteins (see, e.g., Coligan, et al. (2000) Current Protocols in Protein Science, Vols. 1-2, John Wiley and Sons, Inc., NY).

Production, purification, and fragmentation of polyclonal and monoclonal antibodies are described (e.g., Harlow and Lane (1999) Using Antibodies, Cold Spring Harbor Laboratory Press, Cold Spring Harbor, N.Y.); standard techniques for characterizing ligand/receptor interactions are available (see, e.g., Coligan et al. (2001) Current Protocols in Immunology, Vol. 4, John Wiley, Inc., NY); methods for flow cytometry, including fluorescence-activated cell sorting (FACS), are available (see, e.g., Shapiro (2003) Practical Flow Cytometry, John Wiley and Sons, Hoboken, N.J.); and fluorescent reagents suitable for modifying nucleic acids, including nucleic acid primers and probes, polypeptides, and antibodies, for use, for example, as diagnostic reagents, are available (Molecular Probes (2003) Catalogue, Molecular Probes, Inc., Eugene, Oreg.; Sigma-Aldrich (2003) Catalogue, St. Louis, Mo.).

Software packages and databases for determining, e.g., antigenic fragments, leader sequences, protein folding, functional domains, glycosylation sites, and sequence alignments, are available (see, e.g., GCG Wisconsin Package (Accelrys, Inc., San Diego, Calif.); and DeCypher™ (TimeLogic Corp., Crystal Bay, Nev.).

Serum IL-10 concentration levels and exposure levels may be determined by standard methods used in the art. For example, a serum exposure level assay can be performed by collecting whole blood (˜50 μl/mouse) from mouse tail snips into plain capillary tubes, separating serum and blood cells by centrifugation, and determining IL-10 exposure levels by standard ELISA kits and techniques.

Cholesterol tests that directly measure LDL levels can be used and are commercially available (e.g., Beckman Coulter, Inc.; Brea, Calif.: AU System LDL-Cholesterol Test). Traditional tests (which generally use of a colorimetric assay system) for measuring total serum cholesterol are also commercially available and may be used herein (e.g., Sigma-Aldrich, St. Louis, Mo.; BioVision, Inc., Milpitas, Calif.).

When experimental analysis with PEG-IL-10 is desired, a mono-/di-PEG-IL-10 mix described in the patent literature (e.g., US 2011/0250163) can be used. Two exemplary synthetic schemes are as follows:

Exemplary Scheme No. 1.

IL-10 (e.g., rodent or primate) is dialyzed against 50 mM sodium phosphate, 100 mM sodium chloride pH ranges 5-7.4. A 1:1-1:7 molar ratio of 5 kDa PEG-propyladehyde is reacted with IL-10 at a concentration of 1-12 mg/mL in the presence of 0.75-30 mM sodium cyanoborohydride. Alternatively the reaction can be activated with picoline borane in a similar manner. The reaction is incubated at 5-30° C. for 3-24 hours. The pH of the pegylation reaction is adjusted to 6.3, and 7.5 mg/mL of hIL-10 is reacted with PEG to make the ratio of IL-10 to PEG linker 1:3.5. The final concentration of cyanoborohydride is ˜25 mM, and the reaction is carried out at 15° C. for 12-15 hours. The mono- and di-PEG IL-10 are the largest products of the reaction, with the concentration of each at ˜50% at termination. The reaction may be quenched using an amino acid such as glycine or lysine or, alternatively, Tris buffers. Multiple purification methods can be employed such as gel filtration, anion and cation exchange chromatographies, and size exclusion HPLC (SE-HPLC) to isolate the desired pegylated IL-10 molecules.

Exemplary Scheme No. 2.

IL-10 is dialyzed against 10 mM sodium phosphate pH 7.0, 100 mM NaCl. The dialyzed IL-10 is diluted 3.2 times to a concentration of about 0.5 to 12 mg/mL using the dialysis buffer. Prior to the addition of the linker, SC-PEG-12 kDa (Delmar Scientific Laboratories, Maywood, Ill.) and one volume of 100 mM Na-tetraborate at pH 9.1 is added into 9 volumes of the diluted IL-10 to raise the pH of the IL-10 solution to 8.6. The SC-PEG-12K linker is dissolved in the dialysis buffer and the appropriate volume of the linker solution (1.8 to 3.6 mole linker per mole of IL-10) is added into the diluted IL-10 solution to initiate the pegylation reaction. The reaction is carried out at 5° C. in order to control the rate, and the reaction solution is mildly agitated. When the mono-PEG-IL-10 yield, as determined by size exclusion HPLC (SE-HPLC), is close to 40%, the reaction is stopped by adding 1M glycine solution to a final concentration of 30 mM. The pH of the reaction solution is slowly adjusted to 7.0 using an HCl solution, and the reaction is 0.2 micron filtered and stored at −80° C.

### Example 1

### Effect of IL-10 Administered SC and IV on Pharmacodynamic Markers

The pharmacodynamic effects of SC and IV administration of rhIL-10 on total serum cholesterol (TC) and on the primary inflammation markers TNFα and IL-1β were evaluated in human subjects.

Subcutaneous Administration.

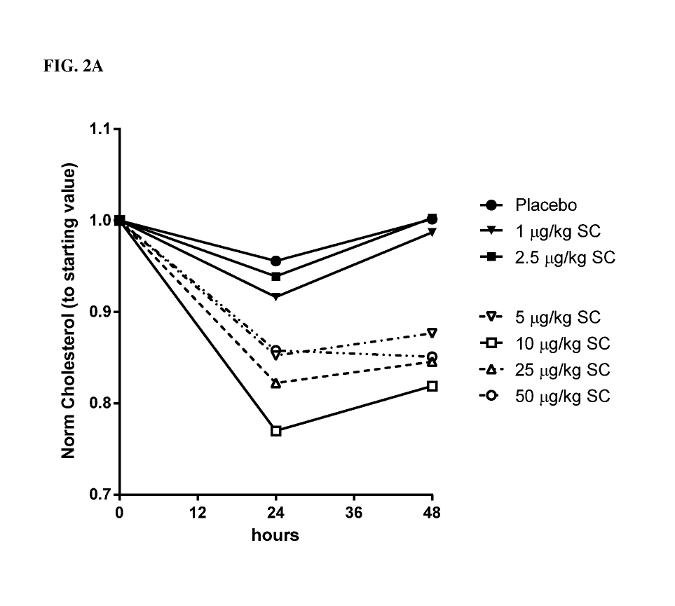

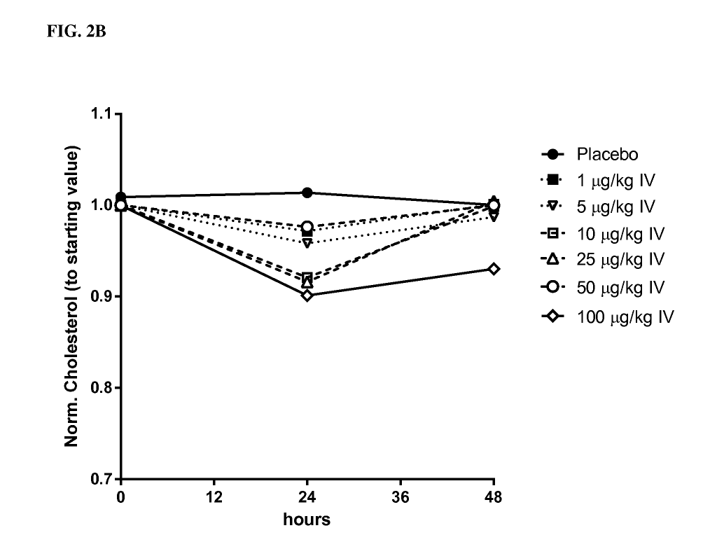

Single ascending doses of rhIL-10 (1.0 μg/kg, 2.5 μg/kg, 5.0 μg/kg, 10 μg/kg, 25 μg/kg and 50 μg/kg) were administered SC to normal, healthy human males (n≧6). TC was measured by conventional techniques 24 hrs and 48 hrs after administration. A 10% reduction in TC 48 hrs after dosing served as the benchmark for a pharmacologically relevant dose. As indicated in FIG. 2A (data are normalized to the starting values) and Table 1, rhIL-10 reduced the concentration of total serum cholesterol by ˜15%-23% at doses of 5 μg/kg, 10 μg/kg, 25 μg/kg and 50 μg/kg. Thus, doses greater than 2.5 μg/kg were pharmacologically relevant 48 hrs post-SC dose. As indicated in Table 1, 2.5 μg/kg resulted in an increase in TC 48 hrs post-SC dose.

TABLE 1

| | | |
|---|---|---|
| | TC Reduction 24 hrs | TC Reduction 48 hrs |
| Dose | Post-SC Dose | Post-SC Dose |

| | | | |
|---|---|---|---|
| 1.0 | μg/kg | 7.9% | 1.3% |
| 2.5 | μg/kg | 6.3% | [0.3% increase] |
| 5 | μg/kg | 14.9% | 12.4% |
| 10 | μg/kg | 22.9% | 18.2% |
| 25 | μg/kg | 17.8% | 15.4% |
| 50 | μg/kg | 14.3% | 14.9% |

Intravenous Administration.

Single ascending doses of rhIL-10 (0.1 μg/kg, 0.5 μg/kg, 1.0 μg/kg, 2.5 μg/kg, 5.0 μg/kg, 10 μg/kg, 25 μg/kg, 50 μg/kg and 100 μg/kg) were also administered IV to normal, healthy human males (n≧6). TC was measured by conventional techniques 24 hrs and 48 hrs after administration. A 10% reduction in TC 48 hrs after dosing served as the benchmark for a pharmacologically relevant dose. As indicated in FIG. 2B and Table 2, rhIL-10 reduced the concentration of total serum cholesterol by ˜8-10% at doses of 10, 25, 50, and 100 μg/kg. As indicated in Table 2, 1.0 μg/kg, 10 μg/kg, and 25 μg/kg resulted in an increase in TC 48 hrs post-IV dose, while 50 μg/kg did not modify TC 48 hrs post-IV dose.

TABLE 2

| | | |
|---|---|---|
| | TC Reduction 24 hrs | TC Reduction 48 hrs |
| Dose | Post-IV Dose | Post-IV Dose |

| | | | |
|---|---|---|---|
| 0.1 | μg/kg | 6.6% | 4.9% |
| 0.5 | μg/kg | NA | 2.3% |
| 1.0 | μg/kg | 3.0% | [0.1% increase] |
| 2.5 | μg/kg | 4.3% | 2.6% |
| 5.0 | μg/kg | 4.2% | 1.3% |
| 10 | μg/kg | 7.8% | [0.1% increase] |
| 25 | μg/kg | 8.4% | [0.45% increase] |
| 50 | μg/kg | 8.6% | [no change] |
| 100 | μg/kg | 10.3% | 7% |

Comparison of Subcutaneous and Intravenous Administration.

As previously indicated, equivalent doses of rhIL-10 administered SC and IV result in different serum concentrations, with SC administration inducing a more pronounced and longer lasting TC reduction than IV administration. As indicated in Tables 1 and 2, at a dose of 5 μg/kg, TC reduction 24 hrs post-SC dose (14.9%) was more than 3-fold greater than TC reduction 24 hrs post-IV dose (4.2%). Moreover, 24 hrs-post dose, 5 μg/kg administered SC would result in a similar TC serum concentration as 25 μg/kg or 50 μg/kg administered IV. Using an ˜10% reduction in TC 24 hrs following IV or SC dosing as a benchmark for a pharmacologically relevant dose, a 5 μg/kg dose of IL-10 administered SC was pharmacologically relevant whereas the same dose administered IV was not. Indeed, the TC reduction observed with administration of 5 μg/kg SC exceeds the TC reduction observed with administration of 5-100 μg/kg IV. This clearly instructs the use of SC administration in the clinical setting described herein. As noted previously, although an understanding of the process underlying this finding is not needed in order to practice the present disclosure, one possible explanation is that SC (but not IV) administration results in the activation of a peripheral mechanism of action (e.g., activation of monocytic, myeloid or lymphocytic compartment).

Inflammation Markers.

In order to determine whether the primary inflammation markers TNFα and IL-1β responded in a qualitative manner to the cholesterol marker, rhIL-10 was administered SC and IV to achieve a serum concentration of approximately 1 ng/mL and reduction in TNFα and IL-1β was measured. In contrast to the data associated with the cholesterol marker, reduction of both TNFα and IL-1β following SC and IV administration of rhIL-10 both tracked the threshold serum concentration of 0.5-1 ng/mL IL-10 (data not shown).

The data set forth above were unexpected because they indicate that SC administration of IL-10 results in greater TC reduction than IV administration of a dose sufficient to achieve the same exposure. This observation is not seen with TNFα and IL-1β. Thus, dosing adjustments can be required when IL-10 or pegylated IL-10 is administered for the treatment of hypercholesterolemia.

Though non-pegylated IL-10 was used in this example, the pharmacodynamic data should be qualitatively predictive of the data that would be generated using PEG-IL-10.

### Example 2

### Cholesterol Reduction Following SC Administration of IL-10

The effect of subcutaneous administration of IL-10 on reduction of total serum cholesterol (TC) was evaluated in human subjects having liver fibrosis resulting from chronic hepatitis C.

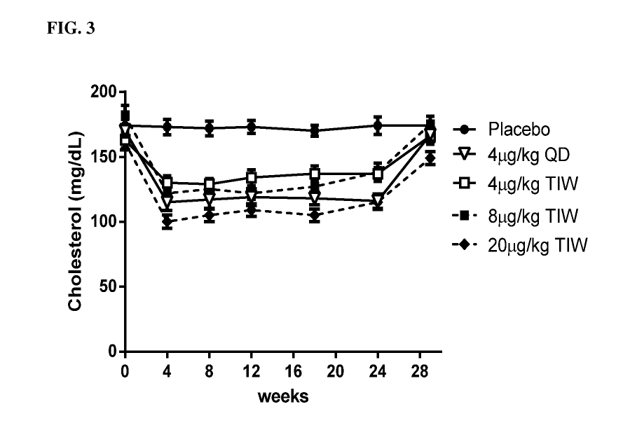

Human subjects were divided into five treatment groups (19-13 males/group): placebo (administered both QD and TIW); 4 μg/kg IL-10 QD; 4 μg/kg IL-10 TIW; 8 μg/kg IL-10 TIW and 20 μg/kg IL-10 TIW. The duration of IL-10 (or placebo) administration for each treatment group was 24 weeks, and TC was measured weekly over a 28-week period. As indicated in FIG. 3, SC administration of 4 μg/kg IL-10 TIW resulted in TC reduction of ≧10% over the duration of the 24 week study. This finding supports the conclusion that a minimum total amount of IL-10 (administered SC) of ˜16 μg/kg/week is required to achieve the ˜10% threshold reduction in TC.

Particular embodiments of this invention are described herein, including the best mode known to the inventors for carrying out the invention. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto, as permitted by applicable law. Moreover, any combination of the above-described elements in all possible variations thereof is encompassed by the invention unless otherwise indicated herein or otherwise clearly contradicted by context.

All publications, patent applications, accession numbers, and other references cited in this specification are herein incorporated by reference as if each individual publication or patent application were specifically and individually indicated to be incorporated by reference.

What is claimed is:

1. A method for determining whether an amount of an IL-10 agent administered subcutaneously is therapeutically effective for treating a cholesterol-related disease, disorder or condition in a subject, comprising
a) establishing a baseline concentration of total serum cholesterol in the subject,
b) administering subcutaneously to the subject a test amount of an IL-10 agent of at least 1 μg/kg, and
c) measuring the total serum cholesterol reduction in the subject at least 24 hours after step b);
wherein a reduction in total serum cholesterol of at least 8% and the absence of an unacceptable indicia of an IL-10 agent—associated toxicity in the subject, indicates a therapeutically effective amount.

2. A method for achieving an amount of an IL-10 agent for subcutaneous administration sufficient for the treatment of a cholesterol-related disease, disorder or condition in a subject, comprising:
a) establishing a baseline concentration of total serum cholesterol in the subject,
b) administering subcutaneously to the subject a first test amount of an IL-10 agent, wherein the first test amount is at least 1 μg/kg,
c) measuring the total serum cholesterol reduction in the subject at least 24 hours after administration of the first test amount, and
wherein a reduction of at least 8% in the subject indicates that the first test amount is sufficient for the treatment of a cholesterol-related disease, disorder or condition in a subject,
wherein a reduction of less than 8% in the subject indicates that the first test amount is suboptimal, whereafter i) a second test amount greater than the first test amount is subcutaneously administered to the subject, wherein the subject has no measurable IL-10 serum concentration, and ii) the total serum concentration in the subject is measured at least 24 hours after the second test amount; wherein steps i) and ii) are repeated until a reduction of at least 8% is achieved indicating that the test amount is sufficient for the treatment of a cholesterol-related disease, disorder or condition.

3. The method of claim 2, wherein the reduction in total serum cholesterol of at least 8% is in the absence of an unacceptable indicia of an IL-10 agent—associated toxicity in the subject.

4. The method of any one of claims 1-3, wherein the measuring of total serum cholesterol reduction is conducted at least 48 hours after step b).

5. The method of any one of claims 1-3, wherein the reduction in total serum cholesterol is at least 10%.

6. The method of any one of claims 1-3, wherein the reduction in total serum cholesterol is at least 12%.

7. The method of any one of claims 1-6, wherein the cholesterol-related disease, disorder or condition is a cardiovascular disorder.

8. The method of claim 7, wherein the cardiovascular disorder is atherosclerosis.

9. The method of claim 7, wherein the subject has hypercholesterolemia or a lipidemia.

10. The method of claim 7, wherein the cardiovascular disorder is a cardiomyopathy.

11. The method of claim 7, wherein the cardiovascular disorder is a hypertensive disorder.

12. The method of any one of claims 1-3, wherein the cholesterol-related disease, disorder or condition is a thrombotic disorder.

13. The method of claim 12, wherein the thrombotic disorder causes stroke or myocardial infarction.

14. The method of any one of claims 1-3, wherein the cholesterol-related disease, disorder or condition is an inflammatory disorder.

15. The method of claim 14, wherein the inflammatory disorder is a vasculitis.

16. The method of any one of claims 1-15, wherein the IL-10 agent is mature human IL-10.

17. The method of any one of claims 1-15, wherein the IL-10 agent is a variant of mature human IL-10, and wherein the variant exhibits activity comparable to the activity of mature human IL-10.

18. The method of any one of claims 1-17, wherein the IL-10 agent comprises at least one modification to form a modified IL-10 agent, wherein the modification does not alter the amino acid sequence of the IL-10 agent.

19. The method of claim 18, wherein the modified IL-10 agent is a pegylated IL-10 agent.

20. The method of claim 19, wherein the pegylated IL-10 agent comprises at least one PEG molecule covalently attached to at least one amino acid residue of at least one subunit of IL-10.

21. The method of claim 19 or 20, wherein the pegylated IL-10 agent comprises a mixture of mono-pegylated and di-pegylated IL-10.

22. The method of any one of claims 19-21, wherein the PEG component of the pegylated IL-10 agent has a molecular mass from about 5 kDa to about 20 kDa.

23. The method of any one of claims 18-22, wherein the modification is site-specific.

24. The method of any one of claims 18-23, wherein the modification comprises a linker.

25. A method of treating a cholesterol-related disease, disorder or condition in a subject, comprising administering subcutaneously to the subject an amount of an IL-10 agent as determined in any one of claims 2-6.

26. A method of treating a cholesterol-related disease, disorder or condition in a subject, comprising administering subcutaneously to the subject an amount of an IL-10 agent of at least 1.0 μg/kg at least every 24 hours.

27. The method of claim 26, comprising administering subcutaneously to the subject an amount of an IL-10 agent of at least 2.0 μg/kg at least every 24 hours.

28. The method of claim 26, comprising administering subcutaneously to the subject an amount of an IL-10 agent of at least 2.0 μg/kg at least every 48 hours.

29. The method of claim 26, comprising administering subcutaneously to the subject an amount of an IL-10 agent of at least 2.5 μg/kg at least every 48 hours.

30. The method of claim 26, comprising administering subcutaneously to the subject an amount of an IL-10 agent of at least 15 μg/kg at least every week.

31. The method of claim 26, comprising administering subcutaneously to the subject an amount of an IL-10 agent of at least 16 μg/kg at least every week.

32. The method of any one of claims 26-31, wherein the amount is sufficient to maintain a mean IL-10 serum trough concentration over a period of time;
wherein the mean IL-10 serum trough concentration is from 0.1 ng/mL to 5.0 ng/mL; and
wherein the mean IL-10 serum trough concentration is maintained for at least 95% of the period of time.

33. The method of claim 32, wherein the mean IL-10 serum trough concentration is greater than 1.0 ng/mL.

34. The method of claim 32, wherein the period of time is at least 48 hours.

35. The method of claim 32, wherein the mean IL-10 serum trough concentration is maintained for 100% of the period of time.

36. The method of any one of claims 26-35, further comprising administering at least one additional prophylactic or therapeutic agent in combination with the IL-10 agent.

37. The method of claim 36, wherein the prophylactic or therapeutic agent is a cholesterol homeostasis agent.

38. The method of claim 37, wherein the cholesterol homeostasis agent comprises a statin, a bile acid resin, ezetimibe, a fibric acid, a niacin, or a PCSK9 inhibitor.

39. The method of claim 36, wherein the prophylactic or therapeutic agent is an anti-diabetic agent or an anti-obesity agent.

40. The method of claim 36, wherein the prophylactic or therapeutic agent is an immune agent or an anti-inflammatory agent.

41. The method of any one of claims 1-40, wherein the subject is a human.

42. A pharmaceutical composition, comprising a pharmaceutically effective amount of an IL-10 agent as determined in any one of claims 2-6, and a pharmaceutically acceptable diluent, carrier or excipient.

43. The pharmaceutical composition of claim 42, wherein the composition is suitable for human administration.

44. The pharmaceutical composition of claim 42 or 43, further comprising at least one additional prophylactic or therapeutic agent.

45. The pharmaceutical composition of claim 44, wherein the prophylactic or therapeutic agent is a cholesterol homeostasis agent, an anti-diabetic agent, an anti-obesity agent, an immune agent, or an anti-inflammatory agent.

46. A sterile container comprising the pharmaceutical composition of any one of claims 42-45.

47. The sterile container of claim 46, wherein the sterile container is a syringe.

48. A kit comprising the sterile container of claim 47.

49. The kit of claim 48, further comprising a second sterile container comprising at least one additional prophylactic or therapeutic agent.

50. A method of determining a therapeutic dosage range of an IL-10 agent for subcutaneous administration to a subject for the treatment of a cholesterol-related disease, disorder or condition, comprising
a) determining a minimum dose of the IL-10 agent, wherein the minimum dose is an amount of the IL-10 agent administered subcutaneously to the subject that provides an approximately 8% reduction in the total serum cholesterol concentration at least 24 hours after the dose compared to a baseline concentration of total serum cholesterol in the subject, and
b) determining a maximum dose of the IL-10 agent, wherein the maximum dose is the highest amount of the IL-10 agent that can be administered subcutaneously to the subject without causing an unacceptable indicia of an IL-10 agent—associated toxicity at a steady state serum level of the IL-10 agent;
wherein the therapeutic dosage range of the IL-10 agent is the minimum dose determined in step a) and the maximum dose determined in step b).

51. A method of determining whether a treatment regimen of an IL-10 agent administered subcutaneously to a subject having a cholesterol-related disease, disorder or condition is suboptimal, comprising
a) determining the total serum cholesterol concentration in the subject at one or more times subsequent to achieving an IL-10 steady state concentration, and
b) comparing the total serum cholesterol concentration determined in step a) with a baseline concentration in the subject determined prior to initiation of the treatment regimen;
wherein a suboptimal dose is an amount of the IL-10 agent insufficient to achieve at least a 8% reduction in the total serum cholesterol in the subject.

52. The method of claim 50 or 51, wherein the reduction in total serum cholesterol is at least 10%.

53. The method of claim 50 or 51, wherein the reduction in total serum cholesterol is at least 12%.


SEQUENCE LISTING (30 sequences)

SEQ ID NO: 1 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - protein, 178 aa, Mus musculus
MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 3 - protein, 11 aa, Artificial Sequence
  Synthetic polypeptide
YGRKKRRQRRR

SEQ ID NO: 4 - protein, 12 aa, Artificial sequence
  Synthetic polypeptide
RRQRRTSKLMKR

SEQ ID NO: 5 - protein, 27 aa, Artificial sequence
  Synthetic polypeptide
GWTLNSAGYLLGKINLKALAALAKKIL

SEQ ID NO: 6 - protein, 33 aa, Artificial sequence
  Synthetic polypeptide
KALAWEAKLAKALAKALAKHLAKALAKALKCEA

SEQ ID NO: 7 - protein, 16 aa, Artificial sequence
  Synthetic polypeptide
RQIKIWFQNRRMKWKK

SEQ ID NO: 8 - protein, 9 aa, Artificial sequence
  Synthetic polypeptide
RKKRRQRRR

SEQ ID NO: 9 - protein, 8 aa, Artificial sequence
  Synthetic polypeptide
RKKRRQRR

SEQ ID NO: 10 - protein, 11 aa, Artificial sequence
  Synthetic polypeptide
YARAAARQARA

SEQ ID NO: 11 - protein, 11 aa, Artificial sequence
  Synthetic polypeptide
THRLPRRRRRR

SEQ ID NO: 12 - protein, 11 aa, Artificial sequence
  Synthetic polypeptide
GGRRARRRRRR

SEQ ID NO: 13 - protein, 5 aa, Artificial sequence
  Synthetic polypeptide
GSGGS

SEQ ID NO: 14 - protein, 4 aa, Artificial sequence
  Synthetic polypeptide
GGGS

SEQ ID NO: 15 - protein, 4 aa, Artificial sequence
  Synthetic polypeptide
GGSG

SEQ ID NO: 16 - protein, 5 aa, Artificial sequence
  Synthetic polypeptide
GGSGG

SEQ ID NO: 17 - protein, 5 aa, Artificial sequence
  Synthetic polypeptide
GSGSG

SEQ ID NO: 18 - protein, 5 aa, Artificial sequence
  Synthetic polypeptide
GSGGG

SEQ ID NO: 19 - protein, 5 aa, Artificial sequence
  Synthetic polypeptide
GGGSG

SEQ ID NO: 20 - protein, 5 aa, Artificial sequence
  Synthetic polypeptide
GSSSG

SEQ ID NO: 21 - protein, 5 aa, Artificial sequence
  Synthetic polypeptide
GSGGS

SEQ ID NO: 22 - protein, 5 aa, Artificial sequence
  Synthetic polypeptide
GSGSG

SEQ ID NO: 23 - protein, 5 aa, Artificial sequence
  Synthetic polypeptide
GSGGS

SEQ ID NO: 24 - protein, 5 aa, Artificial sequence
  Synthetic polypeptide
GSGSG

SEQ ID NO: 25 - protein, 4 aa, Artificial sequence
  Synthetic polypeptide
GGGS

SEQ ID NO: 26 - protein, 5 aa, Artificial sequence
  Synthetic polypeptide
GSGGS

SEQ ID NO: 27 - protein, 4 aa, Artificial sequence
  Synthetic polypeptide
GGGS

SEQ ID NO: 28 - protein, 5 aa, Artificial sequence
  Synthetic polypeptide
GGGGS

SEQ ID NO: 29 - protein, 4 aa, Artificial sequence
  Synthetic polypeptide
GGGS

SEQ ID NO: 30 - protein, 5 aa, Artificial sequence
  Synthetic polypeptide
GGGGS